SEQUENCE LISTING (266 sequences)

SEQ ID NO: 1 - DNA, 22 bp, Artificial Sequence
  oligonucleotide
gcaatctctgcttttgatggct

SEQ ID NO: 2 - DNA, 23 bp, Artificial Sequence
  oligonucleotide
tatgtcttgtggaaacagcactc

SEQ ID NO: 3 - DNA, 22 bp, Artificial Sequence
  oligonucleotide
atggcttctgtggctacagacc

SEQ ID NO: 4 - DNA, 20 bp, Artificial Sequence
  oligonucleotide
aacactgccttccctgaccc

SEQ ID NO: 5 - DNA, 24 bp, Artificial Sequence
  oligonucleotide
gtctaacactgtcctcgctgattc

SEQ ID NO: 6 - DNA, 23 bp, Artificial Sequence
  oligonucleotide
gaaaggtgacattgagctgtcac

SEQ ID NO: 7 - DNA, 26 bp, Artificial Sequence
  oligonucleotide
gaaaaaatcctgatatgcgaacagta

SEQ ID NO: 8 - DNA, 24 bp, Artificial Sequence
  oligonucleotide
caaaaactgaccttgaaatgtcaa

SEQ ID NO: 9 - DNA, 23 bp, Artificial Sequence
  oligonucleotide
agaatgttttgctggaatgtgga

SEQ ID NO: 10 - DNA, 22 bp, Artificial Sequence
  oligonucleotide
tgcttcttgagagcagaaccaa

SEQ ID NO: 11 - DNA, 24 bp, Artificial Sequence
  oligonucleotide
caataatcctgaagtgtgagccag

SEQ ID NO: 12 - DNA, 23 bp, Artificial Sequence
  oligonucleotide
aaggacaaaaagcaaagatgagg

SEQ ID NO: 13 - DNA, 18 bp, Artificial Sequence
  oligonucleotide
cctgggcatgttcttggg

SEQ ID NO: 14 - DNA, 18 bp, Artificial Sequence
  oligonucleotide
tattacttctggggcctg

SEQ ID NO: 15 - DNA, 27 bp, Artificial Sequence
  oligonucleotide
gttggataatttttagtttcttggaag

SEQ ID NO: 16 - DNA, 21 bp, Artificial Sequence
  oligonucleotide
ggccaggaagcagagatgaaa

SEQ ID NO: 17 - DNA, 23 bp, Artificial Sequence
  oligonucleotide
gaaaggtgacattgagctgtcac

SEQ ID NO: 18 - DNA, 24 bp, Artificial Sequence
  oligonucleotide
ggaaaggtgacattgagctgtaat

SEQ ID NO: 19 - DNA, 23 bp, Artificial Sequence
  oligonucleotide
cttctgtcttcttgcagccactt

SEQ ID NO: 20 - DNA, 19 bp, Artificial Sequence
  oligonucleotide
tgcctcttgggaataggcc

SEQ ID NO: 21 - DNA, 26 bp, Artificial Sequence
  oligonucleotide
agtgttctgtctccttgacacagtac

SEQ ID NO: 22 - DNA, 21 bp, Artificial Sequence
  oligonucleotide
cctgctacttctttggagcca

SEQ ID NO: 23 - DNA, 23 bp, Artificial Sequence
  oligonucleotide
gcccagtcgttttatacctgaat

SEQ ID NO: 24 - DNA, 21 bp, Artificial Sequence
  oligonucleotide
gtgctgattacctggccacac

SEQ ID NO: 25 - DNA, 24 bp, Artificial Sequence
  oligonucleotide
gaaaaacgattctctgctgagtgt

SEQ ID NO: 26 - DNA, 24 bp, Artificial Sequence
  oligonucleotide
cttatggacaatcagactgcctca

SEQ ID NO: 27 - DNA, 24 bp, Artificial Sequence
  oligonucleotide
atggagagagataaaggaaacctg

SEQ ID NO: 28 - DNA, 24 bp, Artificial Sequence
  oligonucleotide
gtggagagagacaaaggattccta

SEQ ID NO: 29 - DNA, 23 bp, Artificial Sequence
  oligonucleotide
ggcgatctatctgaaggctatga

SEQ ID NO: 30 - DNA, 25 bp, Artificial Sequence
  oligonucleotide
aaggagacatccctaaaggatacag

SEQ ID NO: 31 - DNA, 19 bp, Artificial Sequence
  oligonucleotide
caaggcctccagaccaagc

SEQ ID NO: 32 - DNA, 20 bp, Artificial Sequence
  oligonucleotide
acaaggccaccagaacaacg

SEQ ID NO: 33 - DNA, 28 bp, Artificial Sequence
  oligonucleotide
ttctactatgataagattttgaacaggg

SEQ ID NO: 34 - DNA, 21 bp, Artificial Sequence
  oligonucleotide
ggcgcttctcacctcagtctt

SEQ ID NO: 35 - DNA, 20 bp, Artificial Sequence
  oligonucleotide
agatgattcagggatgccca

SEQ ID NO: 36 - DNA, 24 bp, Artificial Sequence
  oligonucleotide
caagtctcttatggaagatggtgg

SEQ ID NO: 37 - DNA, 28 bp, Artificial Sequence
  oligonucleotide
gatgaggctgttatagataattcacagt

SEQ ID NO: 38 - DNA, 21 bp, Artificial Sequence
  oligonucleotide
caggtagagtcggtggtgcaa

SEQ ID NO: 39 - DNA, 24 bp, Artificial Sequence
  oligonucleotide
caggaaaaatttcccatcagtcat

SEQ ID NO: 40 - DNA, 28 bp, Artificial Sequence
  oligonucleotide
ggagaagtctaaactgtttaaggatcag

SEQ ID NO: 41 - DNA, 22 bp, Artificial Sequence
  oligonucleotide
aaggacaagtttccaatcagcc

SEQ ID NO: 42 - DNA, 24 bp, Artificial Sequence
  oligonucleotide
ctggttcctttaccaaagaagact

SEQ ID NO: 43 - DNA, 21 bp, Artificial Sequence
  oligonucleotide
ccctgagccgaaggtgtagtc

SEQ ID NO: 44 - DNA, 24 bp, Artificial Sequence
  oligonucleotide
ttctccaaaatagagcgtatttcc

SEQ ID NO: 45 - DNA, 24 bp, Artificial Sequence
  oligonucleotide
ggttccatgaccgaaaaataatct

SEQ ID NO: 46 - DNA, 19 bp, Artificial Sequence
  oligonucleotide
ctccaaaaagcggagcctg

SEQ ID NO: 47 - DNA, 19 bp, Artificial Sequence
  oligonucleotide
cgcaaagtagaggggcgaa

SEQ ID NO: 48 - DNA, 20 bp, Artificial Sequence
  oligonucleotide
ctggtccgaagaactgctca

SEQ ID NO: 49 - DNA, 20 bp, Artificial Sequence
  oligonucleotide
ccaaagtagagctgcccggt

SEQ ID NO: 50 - DNA, 21 bp, Artificial Sequence
  oligonucleotide
cctgagccaaaatacagcgtt

SEQ ID NO: 51 - DNA, 23 bp, Artificial Sequence
  oligonucleotide
gcaccaaagtacaaggtgttttg

SEQ ID NO: 52 - DNA, 20 bp, Artificial Sequence
  oligonucleotide
ggcccaaagtactgggtgtc

SEQ ID NO: 53 - DNA, 19 bp, Artificial Sequence
  oligonucleotide
gccgggaccgaagtactgt

SEQ ID NO: 54 - protein, 22 aa, Mus musculus
  VARIANT 4, 8: Xaa = Ser or Leu, Gly or Ala
CASXDWGXETLYFGSGTRLTVL

SEQ ID NO: 55 - protein, 22 aa, Mus musculus
  VARIANT 4, 8: Xaa = Ser or Ser, Gly or Gln
CASXQDWXNTLYFGAGTRLSVL

SEQ ID NO: 56 - protein, 18 aa, Mus musculus
  VARIANT 4, 7: Xaa = Ser or Ser, Val or Gln
CASXDRXYFGPGTRLTVL

SEQ ID NO: 57 - protein, 21 aa, Mus musculus
  VARIANT 3, 8: Xaa = Ala or Asp,  Ser or Thr
CSXRPGAXEVFFGKGTRLTVV

SEQ ID NO: 58 - protein, 19 aa, Mus musculus
  VARIANT 3, 5: Xaa = Ala or Gly, Thr or Asn
CSXTXERLFFGHGTKLSVL

SEQ ID NO: 59 - protein, 18 aa, Mus musculus
  VARIANT 2, 3: Xaa = Ser or Val, Val or Asn
CXXYAEQFFGPGTRLTVL

SEQ ID NO: 60 - protein, 19 aa, Mus musculus
  VARIANT 3, 5: Xaa = Ala or Asp, Ser or Gln
CSXCXDTQYFGPGTRLLVL

SEQ ID NO: 61 - protein, 19 aa, Mus musculus
  VARIANT 4, 5: Xaa = Ser or Arg, Thr or Asn
CASXXSDYTFGSGTRLLVI

SEQ ID NO: 62 - protein, 20 aa, Mus musculus
  VARIANT 3, 6: Xaa = Gly or Thr, Pro or Asn
CAXGGXERLFFGHGTKLSVL

SEQ ID NO: 63 - protein, 20 aa, Mus musculus
  VARIANT 4, 7: Xaa = Ser or Leu, Arg or Ala
CASXSGXEQFFGPGTRLTVL

SEQ ID NO: 64 - protein, 19 aa, Mus musculus
  VARIANT 4, 7: Xaa = Ser or Leu, Asp or Glu
CASXGDXQYFGPGTRLTVL

SEQ ID NO: 65 - protein, 19 aa, Mus musculus
  VARIANT 4, 5: Xaa = Ser or Gln, Gly or Asn
CASXXSDYTFGSGTRLLVI

SEQ ID NO: 66 - protein, 22 aa, Mus musculus
  VARIANT 4, 7: Xaa = Ser or Leu, Gly or Tyr
CASXERXNERLFFGHGTKLSVL

SEQ ID NO: 67 - protein, 21 aa, Mus musculus
  VARIANT 4, 7: Xaa = Ser or Pro, Gly or Tyr
CASXGLXAEQFFGPGTRLTVL

SEQ ID NO: 68 - protein, 23 aa, Mus musculus
  VARIANT 4, 8: Xaa = Ser or Leu, Gly or Tyr
CASXALGXQNTLYFGAGTRLSVL

SEQ ID NO: 69 - protein, 21 aa, Mus musculus
  VARIANT 4, 8: Xaa = Ser or Leu, Gly or Tyr
CASXAGGXEQQFGPGTRLTVL

SEQ ID NO: 70 - protein, 20 aa, Mus musculus
  VARIANT 4, 8: Xaa = Ser or Phe, Ala or Glu
CASXGQGXVFFGKGTRLTVV

SEQ ID NO: 71 - protein, 19 aa, Mus musculus
  VARIANT 4, 6: Xaa = Ser or Ile, Asp or Ser
CASXRXDYTFGSGTRLLVI

SEQ ID NO: 72 - protein, 21 aa, Mus musculus
  VARIANT 4, 6: Xaa = Ser or Ile, Asp or Asn
CASXRXYAEQFFGPGTRLTVL

SEQ ID NO: 73 - protein, 20 aa, Mus musculus
  VARIANT 4, 6: Xaa = Pro or Gln, Ala or Asn
CASXVXTEVFFGKGTRLTVV

SEQ ID NO: 74 - protein, 20 aa, Mus musculus
  VARIANT 4, 7: Xaa = Ser or Pro, Gly or Asp
CASXGQXTQYFGPGTRLLVL

SEQ ID NO: 75 - protein, 21 aa, Mus musculus
  VARIANT 4, 7: Xaa = Ser or Asp, Asp or Tyr
CASXQGXAEQFFGPGTRLTVL

SEQ ID NO: 76 - protein, 20 aa, Mus musculus
  VARIANT 4, 6: Xaa = Ser or Gly, Gly or Gln
CASXTXDTQYFGPGTRLLVL

SEQ ID NO: 77 - protein, 21 aa, Mus musculus
  VARIANT 4, 7: Xaa = Gly or Val, Gly or Asn
CASXQGXQAPLFGEGTRLSVL

SEQ ID NO: 78 - protein, 22 aa, Mus musculus
  VARIANT 4, 7: Xaa = Gly or Asp, Gly or Ser
CASXLGXAETLYFGSGTRLTVL

SEQ ID NO: 79 - protein, 19 aa, Mus musculus
  VARIANT 4, 7: Xaa = Ser or Asp, Val or Glu
CASXGTXVFFGKGTRLTVV

SEQ ID NO: 80 - protein, 20 aa, Mus musculus
  VARIANT 4, 6: Xaa = Arg or Gly, Ala or Asn
CASXPXSDYTFGSGTRLLVI

SEQ ID NO: 81 - protein, 20 aa, Mus musculus
  VARIANT 4, 5: Xaa = Ser or Glu, Glu or Asn
CASXXTGQLYFGEGSKLTVL

SEQ ID NO: 82 - protein, 21 aa, Mus musculus
  VARIANT 4, 7: Xaa = Ser or Asp, Phe or Gln
CASXWGXNTLYFGAGTRLSVL

SEQ ID NO: 83 - protein, 22 aa, Mus musculus
  VARIANT 4, 9: Xaa = Ser or Arg, Arg or Asp
CASXDTGAXTQYFGPGTRLLVL

SEQ ID NO: 84 - protein, 21 aa, Mus musculus
  VARIANT 4, 6: Xaa = Ser or Gly, Ser or Ser
CASXRXGNTLYFGEGSRLIVV

SEQ ID NO: 85 - protein, 22 aa, Mus musculus
  VARIANT 4, 7: Xaa = Ser or Leu, Arg or Asn
CASXDWXTGQLYFGEGSKLTVL

SEQ ID NO: 86 - protein, 21 aa, Mus musculus
  VARIANT 4, 7: Xaa = Ser or Leu, Ala or Asn
CASXGNXTEVFFGKGTRLTVV

SEQ ID NO: 87 - protein, 21 aa, Mus musculus
  VARIANT 4, 6: Xaa = Ser or Leu, Thr or Ser
CASXGXGNTLYFGEGSRLIVV

SEQ ID NO: 88 - protein, 21 aa, Mus musculus
  VARIANT 4, 7: Xaa = Ser or Pro, Gly or Ala
CASXGTXETLYFGSGTRLTVL

SEQ ID NO: 89 - protein, 22 aa, Mus musculus
  VARIANT 4, 7: Xaa = Ser or Leu, Asp or Ser
CASXEPXQNTLYFGAGTRLSVL

SEQ ID NO: 90 - protein, 19 aa, Mus musculus
  VARIANT 4, 5: Xaa = Ser or Ser, Ser or Asn
CASXXSDYTFGSGTRLLVI

SEQ ID NO: 91 - protein, 20 aa, Mus musculus
  VARIANT 4, 6: Xaa = Ser or Asp, Ala or Gly
CASXRXNTLYFGEGSRLIVV

SEQ ID NO: 92 - protein, 20 aa, Mus musculus
  VARIANT 4, 6: Xaa = Ser or Trp, Gly or Asn
CASXTXQAPLFGEGTRLSVL

SEQ ID NO: 93 - protein, 20 aa, Mus musculus
  VARIANT 4, 5: Xaa = Ser or Leu, Asp or Ser
CASXXQNTLYFGAGTRLSVL

SEQ ID NO: 94 - protein, 19 aa, Mus musculus
  VARIANT 4, 6: Xaa = Ser or Leu, Glu or Asp
CASXYXTQYFGPGTRLLVL

SEQ ID NO: 95 - protein, 21 aa, Mus musculus
  VARIANT 4, 7: Xaa = Ser or Pro, Arg or Asn
CASXRDXTEVFFGKGTRLTVV

SEQ ID NO: 96 - protein, 21 aa, Mus musculus
  VARIANT 4, 7: Xaa = Ser or Leu, Asp or Asn
CASXQGXERLFFGHGTKLSVL

SEQ ID NO: 97 - protein, 21 aa, Mus musculus
  VARIANT 4, 7: Xaa = Ser or Leu, Asp or Gln
CASXWGXDTQYFGPGTRLLVL

SEQ ID NO: 98 - protein, 21 aa, Mus musculus
  VARIANT 4, 7: Xaa = Ser or Pro, Thr or Asn
CAWXPGXTEVFFGKGTRLTVV

SEQ ID NO: 99 - protein, 22 aa, Mus musculus
  VARIANT 4, 9: Xaa = Ser or Leu, Asp or Ser
CAWXPGQGXDYTFGSGTRLLVI

SEQ ID NO: 100 - protein, 21 aa, Mus musculus
  VARIANT 4, 6: Xaa = Arg or Asp, Gln or Asn
CGAXRXYAEQFFGPGTRLTVL

SEQ ID NO: 101 - protein, 19 aa, Mus musculus
  VARIANT 4, 5: Xaa = Arg or Gly, Arg or Gln
CGAXXNTLYFGAGTRLSVL

SEQ ID NO: 102 - protein, 19 aa, Mus musculus
  VARIANT 4, 7: Xaa = Ser or Gln, Lys or Glu
CASXANXVFFGKGTRLTVV

SEQ ID NO: 103 - protein, 21 aa, Mus musculus
  VARIANT 3, 6: Xaa = Ser or Asn, Gly or Tyr
CSXNRXNSPLYFAAGTRLTVT

SEQ ID NO: 104 - protein, 22 aa, Mus musculus
  VARIANT 2, 7: Xaa = Ser or Pro, Ala or Ser
CXRDWGXAETLYFGSGTRLTVL

SEQ ID NO: 105 - protein, 20 aa, Mus musculus
  VARIANT 4, 7: Xaa = Ser or Trp, Ala or Ser
CSSXDRXDYTFGSGTRLLVI

SEQ ID NO: 106 - protein, 21 aa, Mus musculus
  VARIANT 4, 8: Xaa = Ser or Gln, Ile or Gln
CASXEGGXAPLFGEGTRLSVL

SEQ ID NO: 107 - protein, 21 aa, Mus musculus
  VARIANT 4, 8: Xaa = Ser or Gln, Asn or Gln
CASXGGIXNTLYFGAGTRLSV

SEQ ID NO: 108 - protein, 20 aa, Mus musculus
  VARIANT 3, 5: Xaa = Ala or Thr, Val or Ser
CSXEXGNTLYFGEGSRLIVV

SEQ ID NO: 109 - protein, 21 aa, Mus musculus
  VARIANT 2, 6: Xaa = Ser or Gly, Gly or Ser
CXNGQXNERLFFGHGTKLSVL

SEQ ID NO: 110 - protein, 19 aa, Mus musculus
  VARIANT 3, 4: Xaa = Ala or Gln, Gly or Asn
CSXXNQAPLFGEGTRLSVL

SEQ ID NO: 111 - protein, 21 aa, Mus musculus
  VARIANT 3, 6: Xaa = Ala or Gly, Gly or Ser
CRXRRXQNTLYFGAGTRLSVL

SEQ ID NO: 112 - protein, 20 aa, Mus musculus
  VARIANT 4, 6: Xaa = Ser or Leu, Gln or Asn
CASXSXTEVFFGKGTRLTVV

SEQ ID NO: 113 - protein, 20 aa, Mus musculus
  VARIANT 4, 6: Xaa = Ser or Arg, Asp orThr
CASXTXGQLYFGEGSKLTVL

SEQ ID NO: 114 - protein, 19 aa, Mus musculus
  VARIANT 4, 7: Xaa = Ser or Leu, Asp or Ala
CASXNRXQYFGPGTRLTVL

SEQ ID NO: 115 - protein, 21 aa, Mus musculus
  VARIANT 4, 8: Xaa = Ser or Leu, Asp or Ala
CASXNWGXEQFFGPGTRLTVL

SEQ ID NO: 116 - protein, 21 aa, Mus musculus
  VARIANT 4, 7: Xaa = Ser or Leu, Tyr or Thr
CASXSGXGQLYFGEGSKLTVL

SEQ ID NO: 117 - protein, 20 aa, Mus musculus
  VARIANT 4, 5: Xaa = Trp or Gly, Gly or Asn
CASXXQDTQYFGPGTRLLVL

SEQ ID NO: 118 - protein, 21 aa, Mus musculus
  VARIANT 4, 6: Xaa = Ser or Pro, Ser or Asn
CASXDXNQAPLFGEGTRLSVL

SEQ ID NO: 119 - protein, 22 aa, Mus musculus
  VARIANT 4, 7: Xaa = Ser or Leu, Ala or Ser
CASXGGXAETLYFGSGTRLTVL

SEQ ID NO: 120 - protein, 21 aa, Mus musculus
  VARIANT 4, 6: Xaa = Ser or Arg, Val or Asn
CASXTXQDTQYFGPGTRLLVL

SEQ ID NO: 121 - protein, 21 aa, Mus musculus
  VARIANT 4, 8: Xaa = Ser or Met, Ala or Ser
CASXGQEXDYTFGSGTRLLVI

SEQ ID NO: 122 - protein, 20 aa, Mus musculus
  VARIANT 4, 6: Xaa = Ser or Arg, Ser or Tyr
CASXGXAEQFFGPGTRLTVL

SEQ ID NO: 123 - protein, 21 aa, Mus musculus
  VARIANT 4, 7: Xaa = Ser or Leu, Gly or Thr
CASXPGXGQLYFGEGSKLTVL

SEQ ID NO: 124 - protein, 21 aa, Mus musculus
  VARIANT 4, 7: Xaa = Ser or Phe, Ser or Asn
CASXSRXTEVFFGKGTRLTVV

SEQ ID NO: 125 - protein, 20 aa, Mus musculus
  VARIANT 4, 7: Xaa = Ser or Trp, Arg or Tyr
CASXGWXEQYFGPGTRLTVL

SEQ ID NO: 126 - protein, 21 aa, Mus musculus
  VARIANT 4, 7: Xaa = Ser or Asp, Asp or Thr
CASXRSXGQLYFGEGSKLTVL

SEQ ID NO: 127 - protein, 20 aa, Mus musculus
  VARIANT 4, 6: Xaa = Ala or Arg, Thr or Asn
CASXDXERLFFGHGTKLSVL

SEQ ID NO: 128 - protein, 21 aa, Mus musculus
  VARIANT 4, 6: Xaa = Gly or Gly, Thr or Ser
CASXTXAETLYFGSGTRLTVL

SEQ ID NO: 129 - protein, 19 aa, Mus musculus
  VARIANT 4, 6: Xaa = Ser or Asp, His or Ser
CASXAXDYTFGSGTRLLVI

SEQ ID NO: 130 - protein, 20 aa, Mus musculus
  VARIANT 4, 6: Xaa = Ser or Arg, Ser or Asn
CASXEXQAPLFGEGTRLSVL

SEQ ID NO: 131 - protein, 22 aa, Mus musculus
  VARIANT 4, 8: Xaa = Ser or Asp, Ala or Tyr
CASXEDWXAEQFFGPGTRLTVL

SEQ ID NO: 132 - protein, 22 aa, Mus musculus
  VARIANT 4, 8: Xaa = Ser or Thr, Ala or Asn
CASXGGAXERLFFGHGTKLSVL

SEQ ID NO: 133 - protein, 21 aa, Mus musculus
  VARIANT 4, 7: Xaa = Arg or Arg, Arg or Ala
CASXRGXETLYFGSGTRLTVL

SEQ ID NO: 134 - protein, 21 aa, Mus musculus
  VARIANT 4, 6: Xaa = Ser or Asp, Tyr or Asn
CASXRXNQAPLFGEGTRLSVL

SEQ ID NO: 135 - protein, 22 aa, Mus musculus
  VARIANT 4, 6: Xaa = Arg or Arg, Phe or Ser
CASXTXYNSPLYFAAGTRLTVT

SEQ ID NO: 136 - protein, 18 aa, Mus musculus
  VARIANT 4, 5: Xaa = Ser or Tyr, Pro or Tyr
CASXXEQYFGPGTRLTVL

SEQ ID NO: 137 - protein, 21 aa, Mus musculus
  VARIANT 4, 7: Xaa = Ser or Asn, Leu or Gly
CASXRGXNTLYFGEGSRLIVV

SEQ ID NO: 138 - protein, 22 aa, Mus musculus
  VARIANT 4, 9: Xaa = Ser or Leu, Lys or Glu
CASXVERSXRLFFGHGTKLSVL

SEQ ID NO: 139 - protein, 22 aa, Mus musculus
  VARIANT 4, 7: Xaa = Arg or Ala, Ser or Ser
CASXGGXAETLYFGSGTRLTVL

SEQ ID NO: 140 - protein, 20 aa, Mus musculus
  VARIANT 4, 6: Xaa = Ser or Leu, Arg or Asn
CASXGXQAPLFGEGTRLSVL

SEQ ID NO: 141 - protein, 21 aa, Mus musculus
  VARIANT 4, 8: Xaa = Ser or Leu, Asp or Ala
CASXSGGXEQFFGPGTRLTVL

SEQ ID NO: 142 - protein, 20 aa, Mus musculus
  VARIANT 4, 5: Xaa = Ser or Thr, Gln or Ser
CASXXQNTLYFGAGTRLSVL

SEQ ID NO: 143 - protein, 20 aa, Mus musculus
  VARIANT 4, 6: Xaa = Ser or Leu, Gly or Asn
CASXTXSDYTFGSGTRLLVI

SEQ ID NO: 144 - protein, 21 aa, Mus musculus
  VARIANT 4, 7: Xaa = Ser or Phe, Asp or Tyr
CASXWGXAEQFFGPGTRLTVL

SEQ ID NO: 145 - protein, 20 aa, Mus musculus
  VARIANT 4, 6: Xaa = Ser or Phe, Gly or Gln
CASXTXDTQYFGPGTRLLVL

SEQ ID NO: 146 - protein, 21 aa, Mus musculus
  VARIANT 4, 6: Xaa = Arg or Gln, Val or Asn
CAWXRXNQAPLFGEGTRLSVL

SEQ ID NO: 147 - protein, 21 aa, Mus musculus
  VARIANT 4, 5: Xaa = Ser or Arg, Gly or Ser
CAWXXYNSPLYFAAGTRLTVT

SEQ ID NO: 148 - protein, 21 aa, Mus musculus
  VARIANT 4, 6: Xaa = Ser or Arg, Val or Asn
CAWXRXYAEQFFGPGTRLTVL

SEQ ID NO: 149 - protein, 22 aa, Mus musculus
  VARIANT 4, 8: Xaa = Ser or Leu, Ala or Gln
CAWXRLGXDTQYFGPGTRLLVL

SEQ ID NO: 150 - protein, 21 aa, Mus musculus
  VARIANT 3, 7: Xaa = Ala or Arg, Phe or Gly
CGXDRVXNTLYFGEGSRLIVV

SEQ ID NO: 151 - protein, 20 aa, Mus musculus
  VARIANT 4, 6: Xaa = Gly or Gln, Thr or Asn
CGAXGXERLFFGHGTKLSVL

SEQ ID NO: 152 - protein, 20 aa, Mus musculus
  VARIANT 3, 5: Xaa = Ala or Arg, Gly or Tyr
CGXDXNSPLYFAAGTRLTVT

SEQ ID NO: 153 - protein, 20 aa, Mus musculus
  VARIANT 4,6: Xaa = Arg or Asp, Ala or Asn
CSSXSXSKYTFGSGTRLLVI

SEQ ID NO: 154 - protein, 21 aa, Mus musculus
  VARIANT 4, 8: Xaa = Arg or Gly, Gly or Glu
CSSXTGRXRLFFGHGTKLSVL

SEQ ID NO: 155 - protein, 20 aa, Mus musculus
  VARIANT 4, 6: Xaa = Arg or Ala, Ser or Tyr
CSSXNXAEQFFGPGTRLTVL

SEQ ID NO: 156 - protein, 18 aa, Mus musculus
  VARIANT 4, 6: Xaa = Arg or Gly, Cys or Tyr
CSSXGXEQYFGPGTRLTV

SEQ ID NO: 157 - protein, 19 aa, Mus musculus
  VARIANT 4, 6: Xaa = Ser or Leu, Gly or Thr
CSSXQXEVFFGKGTRLTVV

SEQ ID NO: 158 - protein, 21 aa, Mus musculus
  VARIANT 4, 7: Xaa = Ser or Gln, Asp or Asn
CSSXLAXSPLYFAAGTRLTVT

SEQ ID NO: 159 - protein, 21 aa, Mus musculus
  VARIANT 4, 9: Xaa = Ser or Gln, Arg or Asp
CSSXRTGGXTQYFGPGTRLLV

SEQ ID NO: 160 - protein, 11 aa, Mus musculus
CASSRANYEQY

SEQ ID NO: 161 - DNA, 20 bp, Artificial Sequence
  oligonucleotide
gacaggaagtgatcttgcgc

SEQ ID NO: 162 - DNA, 20 bp, Artificial Sequence
  oligonucleotide
aatcttggggcagaaagtcg

SEQ ID NO: 163 - DNA, 29 bp, Artificial Sequence
  oligonucleotide
gataatgtttagctacaataataaggagc

SEQ ID NO: 164 - DNA, 29 bp, Artificial Sequence
  oligonucleotide
gataatgtttagctacaataataaggagc

SEQ ID NO: 165 - DNA, 27 bp, Artificial Sequence
  oligonucleotide
agaagtctttgaaatgtgaacaacata

SEQ ID NO: 166 - DNA, 26 bp, Artificial Sequence
  oligonucleotide
tcatgtttgtctacagctatgagaaa

SEQ ID NO: 167 - DNA, 21 bp, Artificial Sequence
  oligonucleotide
aacaacatctggggcataacg

SEQ ID NO: 168 - DNA, 27 bp, Artificial Sequence
  oligonucleotide
gagctcatgtttgtctacaactttaaa

SEQ ID NO: 169 - DNA, 21 bp, Artificial Sequence
  oligonucleotide
ccctatctctgggcataggag

SEQ ID NO: 170 - DNA, 26 bp, Artificial Sequence
  oligonucleotide
agctcatgtttgtctacagtcttgaa

SEQ ID NO: 171 - DNA, 22 bp, Artificial Sequence
  oligonucleotide
cttctcagtctgggcacaacac

SEQ ID NO: 172 - DNA, 26 bp, Artificial Sequence
  oligonucleotide
ttcctctttgaatacttcagtgagac

SEQ ID NO: 173 - DNA, 23 bp, Artificial Sequence
  oligonucleotide
tctcctatctctgggcacaagag

SEQ ID NO: 174 - DNA, 28 bp, Artificial Sequence
  oligonucleotide
cagtttatctttcagtattatagggagg

SEQ ID NO: 175 - DNA, 22 bp, Artificial Sequence
  oligonucleotide
tctcctaagtctgggcatgaca

SEQ ID NO: 176 - DNA, 28 bp, Artificial Sequence
  oligonucleotide
cccagtttatctttcagtattatgagaa

SEQ ID NO: 177 - DNA, 21 bp, Artificial Sequence
  oligonucleotide
cctatctctgggcacaccagt

SEQ ID NO: 178 - DNA, 28 bp, Artificial Sequence
  oligonucleotide
ccagtttatctttcagtattatgaggag

SEQ ID NO: 179 - DNA, 23 bp, Artificial Sequence
  oligonucleotide
tgcccaggatatgaaccataact

SEQ ID NO: 180 - DNA, 20 bp, Artificial Sequence
  oligonucleotide
cctttggtatgacgagggtg

SEQ ID NO: 181 - DNA, 21 bp, Artificial Sequence
  oligonucleotide
gcccaggatatgaaccatgaa

SEQ ID NO: 182 - DNA, 25 bp, Artificial Sequence
  oligonucleotide
gatttattactcagcttctgagggt

SEQ ID NO: 183 - DNA, 27 bp, Artificial Sequence
  oligonucleotide
agatgtacccaggatatgagacataat

SEQ ID NO: 184 - DNA, 25 bp, Artificial Sequence
  oligonucleotide
gctgattcattactcagttggtgag

SEQ ID NO: 185 - DNA, 26 bp, Artificial Sequence
  oligonucleotide
tgtacccaggatatgaaccataacta

SEQ ID NO: 186 - DNA, 26 bp, Artificial Sequence
  oligonucleotide
ctaaggctcatccattattcaaatac

SEQ ID NO: 187 - DNA, 21 bp, Artificial Sequence
  oligonucleotide
cccaggatatgaaccatggat

SEQ ID NO: 188 - DNA, 24 bp, Artificial Sequence
  oligonucleotide
ctgattcattactcagttggtgct

SEQ ID NO: 189 - DNA, 24 bp, Artificial Sequence
  oligonucleotide
ggtgtgatccaatttcaggtcata

SEQ ID NO: 190 - DNA, 22 bp, Artificial Sequence
  oligonucleotide
ggggctgaagctgatttattat

SEQ ID NO: 191 - DNA, 22 bp, Artificial Sequence
  oligonucleotide
ttcaatttcgggtcatgtaacc

SEQ ID NO: 192 - DNA, 23 bp, Artificial Sequence
  oligonucleotide
actactcagctgctgctggtact

SEQ ID NO: 193 - DNA, 21 bp, Artificial Sequence
  oligonucleotide
ccaatttcgggtcatgtatcc

SEQ ID NO: 194 - DNA, 16 bp, Artificial Sequence
  oligonucleotide
gcatggggctgaggcg

SEQ ID NO: 195 - DNA, 22 bp, Artificial Sequence
  oligonucleotide
gatccaatttcgagtcatgcaa

SEQ ID NO: 196 - DNA, 24 bp, Artificial Sequence
  oligonucleotide
ttttaatttacttccaaggcaaca

SEQ ID NO: 197 - DNA, 22 bp, Artificial Sequence
  oligonucleotide
atccaatttctgaacacaaccg

SEQ ID NO: 198 - DNA, 23 bp, Artificial Sequence
  oligonucleotide
ttctaatttacttccaaggcacg

SEQ ID NO: 199 - DNA, 21 bp, Artificial Sequence
  oligonucleotide
tgctcccctaggtctggagac

SEQ ID NO: 200 - DNA, 24 bp, Artificial Sequence
  oligonucleotide
ggttctgacttactcccagagtga

SEQ ID NO: 201 - DNA, 23 bp, Artificial Sequence
  oligonucleotide
accagacttggaaccacaacaat

SEQ ID NO: 202 - DNA, 24 bp, Artificial Sequence
  oligonucleotide
tgacttacttcaattatgaagccc

SEQ ID NO: 203 - DNA, 22 bp, Artificial Sequence
  oligonucleotide
ccagacttggagccacagctat

SEQ ID NO: 204 - DNA, 27 bp, Artificial Sequence
  oligonucleotide
cccagagtttctgacttacttcaatta

SEQ ID NO: 205 - DNA, 20 bp, Artificial Sequence
  oligonucleotide
accagactgagaaccaccgc

SEQ ID NO: 206 - DNA, 25 bp, Artificial Sequence
  oligonucleotide
ctgacttatttccagaatgaagctc

SEQ ID NO: 207 - DNA, 22 bp, Artificial Sequence
  oligonucleotide
tggctttttggtgtgatcctat

SEQ ID NO: 208 - DNA, 22 bp, Artificial Sequence
  oligonucleotide
gggcccagagtttctgacttac

SEQ ID NO: 209 - DNA, 22 bp, Artificial Sequence
  oligonucleotide
ggctttttggtgcaatcctata

SEQ ID NO: 210 - DNA, 27 bp, Artificial Sequence
  oligonucleotide
cagttcctcattcagtattataatgga

SEQ ID NO: 211 - DNA, 22 bp, Artificial Sequence
  oligonucleotide
ggctttttggtgcaatcctatt

SEQ ID NO: 212 - DNA, 25 bp, Artificial Sequence
  oligonucleotide
tccattactcatatggtgttcaaga

SEQ ID NO: 213 - DNA, 21 bp, Artificial Sequence
  oligonucleotide
accaatttcaggccacaactc

SEQ ID NO: 214 - DNA, 26 bp, Artificial Sequence
  oligonucleotide
tctattactcagcagctgctgatatt

SEQ ID NO: 215 - DNA, 25 bp, Artificial Sequence
  oligonucleotide
gtaaaccaatttcaggacacgacta

SEQ ID NO: 216 - DNA, 27 bp, Artificial Sequence
  oligonucleotide
gatccattactcatatggtgttaaaga

SEQ ID NO: 217 - DNA, 23 bp, Artificial Sequence
  oligonucleotide
cagccaattttaggccacaatac

SEQ ID NO: 218 - DNA, 19 bp, Artificial Sequence
  oligonucleotide
cccggagcttctggttcaa

SEQ ID NO: 219 - DNA, 24 bp, Artificial Sequence
  oligonucleotide
ccactctgaaatgctatcctatcc

SEQ ID NO: 220 - DNA, 23 bp, Artificial Sequence
  oligonucleotide
ccaaagcttctgattcagtttca

SEQ ID NO: 221 - DNA, 24 bp, Artificial Sequence
  oligonucleotide
gacccaatttctggacatgataat

SEQ ID NO: 222 - DNA, 24 bp, Artificial Sequence
  oligonucleotide
gattcgatatgagaatgaggaagc

SEQ ID NO: 223 - DNA, 25 bp, Artificial Sequence
  oligonucleotide
gttctcagactttgaaccataacgt

SEQ ID NO: 224 - DNA, 24 bp, Artificial Sequence
  oligonucleotide
atttactttaacaacaacgttccg

SEQ ID NO: 225 - DNA, 24 bp, Artificial Sequence
  oligonucleotide
caaaattatattgtgccccaataa

SEQ ID NO: 226 - DNA, 24 bp, Artificial Sequence
  oligonucleotide
atttactttaacaacaacgttccg

SEQ ID NO: 227 - DNA, 22 bp, Artificial Sequence
  oligonucleotide
cccaatgaaaggacacagtcat

SEQ ID NO: 228 - DNA, 17 bp, Artificial Sequence
  oligonucleotide
acttccgcaaccgggct

SEQ ID NO: 229 - DNA, 23 bp, Artificial Sequence
  oligonucleotide
tgaacagaatttgaaccacgatg

SEQ ID NO: 230 - DNA, 24 bp, Artificial Sequence
  oligonucleotide
atttcgttttatgaaaagatgcag

SEQ ID NO: 231 - DNA, 21 bp, Artificial Sequence
  oligonucleotide
ctgtgaagatcgagtgccgtt

SEQ ID NO: 232 - DNA, 28 bp, Artificial Sequence
  oligonucleotide
tctgttacattttgtgaaagagtctaaa

SEQ ID NO: 233 - DNA, 26 bp, Artificial Sequence
  oligonucleotide
tgttctcagactaagggtcatgatag

SEQ ID NO: 234 - DNA, 24 bp, Artificial Sequence
  oligonucleotide
aaagctgctgttccactactatga

SEQ ID NO: 235 - DNA, 23 bp, Artificial Sequence
  oligonucleotide
cactctggaatgttctcaaacca

SEQ ID NO: 236 - DNA, 24 bp, Artificial Sequence
  oligonucleotide
atttccttccagaatgaaaatgtc

SEQ ID NO: 237 - DNA, 28 bp, Artificial Sequence
  oligonucleotide
ttgttctcagaatatgaaccatgagtat

SEQ ID NO: 238 - DNA, 27 bp, Artificial Sequence
  oligonucleotide
atggtttatctccagaaagaaaatatc

SEQ ID NO: 239 - DNA, 22 bp, Artificial Sequence
  oligonucleotide
ggaatgtgtccaggatatggac

SEQ ID NO: 240 - DNA, 31 bp, Artificial Sequence
  oligonucleotide
attgatctactactcacagatagtaaatgac

SEQ ID NO: 241 - DNA, 22 bp, Artificial Sequence
  oligonucleotide
aagtcgatagccaagtcaccat

SEQ ID NO: 242 - DNA, 20 bp, Artificial Sequence
  oligonucleotide
caacttccaatgagggctcc

SEQ ID NO: 243 - DNA, 20 bp, Artificial Sequence
  oligonucleotide
gtggagggaacatcaaaccc

SEQ ID NO: 244 - DNA, 24 bp, Artificial Sequence
  oligonucleotide
gcctacggttgatctattactcct

SEQ ID NO: 245 - DNA, 26 bp, Artificial Sequence
  oligonucleotide
ctcatccactattcctatggagttaa

SEQ ID NO: 246 - DNA, 27 bp, Artificial Sequence
  oligonucleotide
agatctactattcaatgaatgttgagg

SEQ ID NO: 247 - DNA, 30 bp, Artificial Sequence
  oligonucleotide
gctgatctatttctcatatgatgttaaaat

SEQ ID NO: 248 - DNA, 21 bp, Artificial Sequence
  oligonucleotide
tgacactgatcgcaactgcaa

SEQ ID NO: 249 - DNA, 22 bp, Artificial Sequence
  oligonucleotide
cttctactccgttggtattggc

SEQ ID NO: 250 - DNA, 20 bp, Artificial Sequence
  oligonucleotide
tgccttgtccaaagaaagct

SEQ ID NO: 251 - DNA, 19 bp, Artificial Sequence
  oligonucleotide
cgaaccgaaggtgtagcca

SEQ ID NO: 252 - DNA, 25 bp, Artificial Sequence
  oligonucleotide
aacttccctctccaaaatatatggt

SEQ ID NO: 253 - DNA, 22 bp, Artificial Sequence
  oligonucleotide
ttccactgccaaaaaacagttt

SEQ ID NO: 254 - DNA, 18 bp, Artificial Sequence
  oligonucleotide
accaaaatgctggggctg

SEQ ID NO: 255 - DNA, 19 bp, Artificial Sequence
  oligonucleotide
cccaaagtggaggggtgaa

SEQ ID NO: 256 - protein, 20 aa, Artificial Sequence
  oligonucleotide
CCCGAAGAACTGCTCATTGT

SEQ ID NO: 257 - DNA, 21 bp, Artificial Sequence
  oligonucleotide
ccttctccaaaaaacagctcc

SEQ ID NO: 258 - DNA, 20 bp, Artificial Sequence
  oligonucleotide
ctgggccaaaatactgcgta

SEQ ID NO: 259 - DNA, 19 bp, Artificial Sequence
  oligonucleotide
cggcgccgaagtactgaat

SEQ ID NO: 260 - DNA, 19 bp, Artificial Sequence
  oligonucleotide
tggcccgaagtactgggtc

SEQ ID NO: 261 - DNA, 18 bp, Artificial Sequence
  oligonucleotide
gccccgaaagtcaggacg

SEQ ID NO: 262 - DNA, 20 bp, Artificial Sequence
  oligonucleotide
ggcccgaagtactgctcgta

SEQ ID NO: 263 - DNA, 20 bp, Artificial Sequence
  oligonucleotide
ccgaggtcgctgtgtttgag

SEQ ID NO: 264 - DNA, 20 bp, Artificial Sequence
  oligonucleotide
ggacttgacagccgaagtgg

SEQ ID NO: 265 - DNA, 20 bp, Artificial Sequence
  oligonucleotide
caaaaggccacactggtgtg

SEQ ID NO: 266 - DNA, 21 bp, Artificial Sequence
  oligonucleotide
ctgctcaggcagtatctggag

(19) United States
(12) Patent Application Publication
(10) Pub. No.: US 2010/0285984 A1
(43) Pub. Date: Nov. 11, 2010

(54) ASSESSING T CELL REPERTOIRES

(76) Inventors: Peter J. Wettstein, Rochester, MN (US); Nancy D. Borson, Rochester, MN (US); Michael A. Strausbauch, Rochester, MN (US)

Correspondence Address: FISH & RICHARDSON P.C., PO BOX 1022, MINNEAPOLIS, MN, 55440-1022, US

(21) Appl. No.: 12/680,065

(22) PCT Filed: Sep. 26, 2008
(86) PCT No.: PCT/US08/77868

§ 371 Date: Jul. 2, 2010

Related U.S. Application Data
(60) Provisional application No. 60/975,980, filed on Sep. 28, 2007.

(51) Int. Cl. C40B 30/04; C12Q 1/68

(52) U.S. Cl. 506/9; 435/6

(57) ABSTRACT

This document provides methods and materials related to assessing T cell repertoires. For example, amplification methods and materials that can be used to assess the diversity of a mammal's T cell repertoire are provided.

### BACKGROUND

1. Technical Field

This document relates to methods and materials involved in assessing T cell repertoires.

2. Background Information

The diversity of T cell repertoires is dependent on the range of combinations of unique alpha and beta subunits that determine the antigenic specificity of T cell receptors (TcRs). This specificity is determined by the utilized variable (V) and joining (J) regions in alpha and beta subunits as well as the diversity (D) regions in beta subunits (Chien et al., Nature, 309:322-326 (1984)). Recombinations between V and J gene segments can result in the formation of complementarity-determining region 3s (CDR3s) that include the carboxy and amino termini of the V and J segments, respectively, as well as variable numbers of random nucleotides inserted between the V and J segments. CDR3s can impact antigenic specificity through their lengths and amino acid sequences (McHeyzer-Williams and Davis, Science, 268:106-111 (1995); Kedzierska et al., Proc. Natl. Acad. Sci. USA, 102:11432-11437 (2005); McHeyzer-Williams et al., J. Exp. Med., 189:1823-1837 (1999); and Zhong and Reinherz, Intl. Immunol., 16:1549-1559 (2004)) that contact the amino and carboxy termini of peptides that are bound to the products of major histocompatibility complex (MHC) class I and class II genes (Garcia et al., Science, 279:1166-1172 (1998)).

### SUMMARY

This document provides methods and materials related to assessing T cell repertoires. For example, this document provides amplification methods and materials that can be used to assess the diversity of a mammal's T cell repertoire. Such methods and materials can provide a unified platform for evaluating repertoire diversity and identifying prominent beta transcripts. The methods and materials provided herein can be based, in part, on the amplification of transcripts carrying individual BV-BJ combinations. In some cases, the simultaneous amplification of all possible BV-BJ combinations by real-time PCR can yield quantitative endpoints for comparisons of repertoire diversity. The increased dissection of populations of beta transcripts can greatly increase the numbers of sequences that can be obtained from selected T cell populations.

In general, one aspect of this document features a method for assessing T cell receptor diversity in a mammal. The method comprises performing a real-time amplification reaction using a BV-specific primer, a BJ-specific primer, and sample of nucleic acid containing template, wherein the sample is enriched to contain BV-BC nucleic acid sequences. The mammal can be a human. The sample can be a sample that was enriched using an amplification reaction that amplifies BV-BC nucleic acid sequences. The amplification reaction that amplifies BV-BC nucleic acid sequences can comprise using an outer BV-specific primer and a BC-specific primer, wherein one of the outer BV-specific primer and the BC-specific primer comprises a label. The label can comprise biotin. Streptavidin-containing magnetic particles can be used to enrich the sample. The method can comprise performing the real-time amplification reaction using a collection of different BV-specific primers, a collection of different BJ-specific primers, and the sample. The collection of different BV-specific primers can comprise a primer specific for each BV nucleic acid present is the mammal. The collection of different BJ-specific primers can comprise a primer specific for each BJ nucleic acid present is the mammal. The sample can be a sample that was enriched using pools of amplification reactions that amplify BV-BC nucleic acid sequences.

The methods and materials provided herein for the beta locus of T cells can be applied to the alpha, gamma, and/or delta loci. For example, the diversity of gamma, delta T cells can be determined using amplification reactions with primer pair specific for either the gamma locus or delta locus.

Another aspect of this document features a method for assessing T cell receptor diversity in a mammal. The method comprises performing a real-time amplification reaction using a GammaV-specific primer, a GammaJ-specific primer, and sample of nucleic acid containing template, wherein the sample is enriched to contain GammaV-GammaC nucleic acid sequences.

Another aspect of this document features a method for assessing T cell receptor diversity in a mammal. The method comprises performing a real-time amplification reaction using a DeltaV-specific primer, a DeltaJ-specific primer, and sample of nucleic acid containing template, wherein the sample is enriched to contain DeltaV-DeltaC nucleic acid sequences.

Another aspect of this document features a method for assessing T cell receptor diversity in a mammal. The method comprises performing a real-time amplification reaction using a AlphaV-specific primer, a AlphaJ-specific primer, and sample of nucleic acid containing template, wherein the sample is enriched to contain AlphaV-AlphaC nucleic acid sequences.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic diagram of RT-PCRs and BV-BJ-specific real-time PCRs.

FIG. 2 contains representative graphs of the uptake of SYBR Green during BV-BJ-specific amplification of beta transcripts expressed by normal mouse lymphocytes. Data are presented for real-time PCRs that were performed with the noted three BV-specific forward primers paired with all 12 BJ-specific reverse primers. The bold horizontal line is the threshold used for calculating Ct values.

FIG. 3 contains representative dissociation curves for products amplified in BV-BJ-specific real-time PCRs from beta transcripts expressed by normal mouse lymphocytes. The presented data are derived from the real-time PCRs described in FIG. 2.

FIG. 4. Representations of 240 BV-BJ combinations in RNA templates extracted from normal and immunodeficient mouse spleens. (A) Ct values for individual BV-BJ-specific amplifications and (B) frequency distributions of Ct values for RNA samples extracted from normal spleens.

FIG. 5. Effects of titration of RNA template on amplification of beta transcripts carrying 240 BV-BJ combinations. (A) Ct values for individual BV-BJ-specific amplifications and (B) frequency distributions of Ct values for titrated RNA samples.

FIG. 6. Representations of 240 BV-BJ combinations in RNA templates extracted from H4-incompatible skin grafts at the time of rejection by two recipients. (A) Ct values for individual BV-BJ-specific amplifications and (B) frequency distributions of Ct values.

FIG. 7. Representations of 240 BV-BJ combinations in RNA templates extracted from HY-incompatible skin grafts at the time of rejection by two female recipients. (A) Ct values for individual BV-BJ-specific amplifications and (B) frequency distributions of Ct values.

FIG. 8. Representative comparisons of dissociation curves for products of BV-BJ-specific amplifications of RNA templates from normal lymphocytes and lymphocytes infiltrating H4- and HY-incompatible skin grafts.

FIG. 9. Human BV-BJ Matrix. Speed of amplification correlates with gray scale.

FIG. 10. Distribution of Ct values from 240 BV-BJ combinations and reproducibility of BV-BJ-specific amplification. (A) Representation of the distribution of Ct values using total RNA template extracted from normal B6 splenocytes. (B-D) Histograms of the distributions of Ct values from Panel A (B) and two replicate amplifications (C and D) of the same RNA template with notations of the mean Ct values and 95% confidence intervals (CIs) of the means. (E) Mean ΔCt values and 95% CIs for pairwise comparisons of Ct values from 240 BV-BJ combinations in the three replicate amplifications.

FIG. 11. Effects of decreasing numbers of RT-PCR cycles on mean Ct values in BV-BJ real-time PCRs. (A) Distribution of Ct values following 25 cycles of RT-PCR and (B) distribution of Ct values following 20 cycles of RT-PCR.

FIG. 12. Effects of titration of products of pooled RT-PCRs on BV-BJ-specific amplification in real-time PCRs. Beta transcripts in total RNA from B6 splenocytes were amplified in pooled RT-PCRs, and specific amplicons were enriched with magnetic beads. Enriched products were amplified in BV-BJ-specific real-time reactions after either no dilution (Panel A) or dilutions of ¼ (Panel B) and 1/16 (Panel C). Comparisons of Ct values from matched BV-BJ-specific amplifications were performed to yield distributions of ΔCt values and estimations of mean Δt values (Panels D and E).

FIG. 13. Effects of titration of RNA template on BV-BJ-specific amplification of beta transcripts. Total RNA template from B6 splenocytes was amplified in pooled RT-PCRs after no dilution or dilutions of ¼ and 1/16. Specific amplicons were enriched with magnetic beads and amplified with BV-BJ primer pairs in real-time PCRs. Distributions of Ct values are presented in Panels A (undiluted), B (¼ dilution), and C ( 1/16 dilution). Comparisons of Ct values from matched BV-BJ-specific amplifications were performed to yield distributions of ΔCt values and estimations of mean ΔCt values (Panels D and E).

FIG. 14. Representations of 240 BV-BJ combinations in RNA extracted from normal and immunodeficient mouse spleen cells. Total RNA was extracted from normal B10 splenocytes (Panel A), B cell-depleted nude mouse splenocytes (Panel B), and NOD-scid spleen cells (Panel C), and amplified by the BV-BJ matrix method. Amplicons with relatively narrow dissociation curves were selected for direct sequencing and the translations of single copy sequences are presented. Some of the results re-presented in FIG. 14 are presented in FIG. 4.

FIG. 15. Detection of an experimentally over-represented transcript by the BV-BJ matrix method. Normal B6 and transgenic OT-1 spleen cells were mixed in a 100:1 ratio prior to the extraction of total RNA. Total RNA template was amplified by the BV-BJ matrix method and the dissociation curves of products amplified by pairings of the BV5.2 primer with the 12 BJ primers are presented. The BV5.2-BJ2.7 amplicons were directly sequenced and the translated sequence matched the CDR3 of the OT-1 beta chain.

### DETAILED DESCRIPTION

The majority of scientific evidence points toward the importance of diversity in T cell repertoires for maintaining memory responses to recall antigens and initiating responses to previously unencountered pathogens and tumors. There are many diseases (AIDS), conditions (aging), and clinical treatments that can reduce the size and potentially the diversity of T cell compartments. These treatments include chemotherapy, radiation therapy, and pretreatment of recipients prior to bone marrow and stem cell transplants.

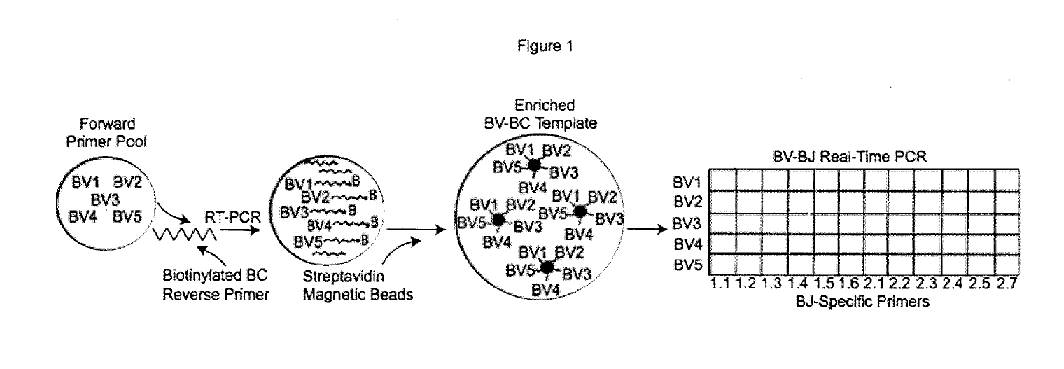

Methods and materials are provided herein for repertoire analysis that can overcome limitations to current technologies. The methods and materials provided herein can include evaluating beta transcript repertoires by subdividing the repertoire into all, or substantially all (e.g., 75 percent, 80 percent, 85 percent, 90 percent, 95 percent, 99 percent, or more), BV-BJ combinations for simultaneous amplifications by real-time PCR (FIG. 1). There are 240 and 611 BV-BJ combinations in mice and humans, respectively, that provide previously unattainable resolution. Specificity can be achieved in part by selection of BJ and nested BV primers. The methods can involve amplification in first-stage RT-PCRs that use pools of BV primers and a single constant region primer labeled with, for example, biotin. The pooled amplicons from beta transcripts can be enriched by binding to, for example, streptavidin-coated magnetic beads to increase specificity in subsequent real-time PCRs. Pooled products can then be aliquoted into wells with individual combinations of nested BV and BJ primers for amplification in real-time PCRs. Amplification can be monitored by uptake of SYBR Green dye, and the tempo of amplification in each well can be estimated by the number of cycles (Ct) required to reach a defined threshold. Specific amplification of beta transcripts can be monitored by dissociation curves. Estimation of Ct values and generation of dissociation curves can be standard processes in real-time PCRs so no additional data analysis may be required. Comparisons of repertoire diversities in different samples can be performed with Wilcoxon matched pairs tests in a straight-forward statistical analysis. This simplified analysis is possible because the BV-BJ combinations can be defined by individual and specific primer pairs whereas the distributions of CDR3 lengths generated by spectratyping can be highly variable. The relatively large BV-BJ matrices can increase the rate of success in identifying and sequencing over-represented beta transcripts relative to spectratyping where amplification can be specific for BV genes alone. In summary, the methods and materials described herein can provide an approach with a integration of individual methods for comprehensive analysis of T cell repertoires. The methods and materials provided herein can be based on (1) particular pairs of BV primers that facilitate the specific amplification of all, or substantially all, expressed BV genes in a mammal (e.g., a mouse or human), (2) BV and BJ primers with increased melting temperatures to promote specific amplifications, (3) DNase treatment of template to eliminate contaminating DNA templates, (4) pooled RT-PCRs using multiple BV-specific primers to reduce required amounts of template, supplies, and labor, (5) enrichment of RT-PCR products of beta transcripts with streptavidin-conjugated magnetic beads to increase specificity, (6) use of fully nested real-time PCRs for quantitation of amounts of templates through estimations of Ct values, (7) use of SYBR Green to monitor amplification of beta transcripts rather than fluorochrome-labeled primers, (8) paired statistical analysis to reduce effects of variable primer efficiency and variable expression of individual BV genes, and (9) increased numbers of sequences that can be obtained without cloning amplified products.

As described herein, the methods and materials provided herein can be used to assess the diversity of a mammal's T cell repertoire. In some cases, the methods and materials provided herein can be used to evaluate TcR diversity in individuals with compromised or reconstituted immune systems. In some cases, the methods and materials provided herein can be used analyze T cell populations that infiltrate sites of autoimmunity, transplant rejection, and tumors, in order to provide information on the diversity and specificity of infiltrating T cells.

A BV-BJ matrix method can be designed to analyze efficiently the diversities of beta transcript repertoires and maximize identification and sequencing of over-represented beta transcripts. The utilization of real-time PCR instrumentation for analysis of TcR repertoires can offer a number of improvements in sample handling, data acquisition, and data analysis. First, the simultaneous monitoring of amplification in all reactions through incorporation of SYBR Green can provide estimates of the tempo of amplification throughout the entire reactions with quantitative endpoints (Ct values). Second, automated melting at the completion of the reactions can provide dissociation curves which can be used to confirm specific amplification. These automated analyses can eliminate the additional sample handling and electrophoretic separation required in spectratyping for identification, separation, and quantitation of products. Third, the simultaneous analysis of amplification with a single matrix of BV-BJ primer pairs simplifies data organization and statistical analysis. The dissection of beta transcript repertoires with a matrix of defined BV-BJ combinations allowed one to estimate relative beta transcript diversities by Shannon entropy, which has been used to estimate the variability of individual amino acid positions in the variable regions of immunoglobulin heavy chains and TcR beta chains (Litwin and Jores, In Perelson and Weisbuch (ed.) Theoretical and experimental insights into immunology, Springer-Verlag, Berlin (1992) and Stewart et al., Mol. Immunol., 34:1067-1082 (1997)). The relatively large number of BV-BJ primer pairs increases the sensitivity of Shannon entropy (Shannon, The Bell System Technical Journal, 27:379-423 & 623-656 (1948)), and continuous Ct values, rather than simple “presence” or “absence” of amplification, increase the amount of information in these diversity estimates. Fourth, the increased resolution associated with matrices of, for example, 240 BV-BJ combinations can improve the efficiency of identifying and sequencing over-represented transcripts due to the increased number of individual PCRs that increases the probability of obtaining products derived predominantly from single beta transcripts.

In some cases, representation of combinations of BV and BJ genes can be less affected by prior exposures to antigens due to their more limited, direct roles in peptide recognition so amplification with BV-BJ primer pairs can yield more unbiased estimates of repertoire diversity.

Analysis of CDR3 length restriction can provide important information on potential skewing of repertoires due to in vivo priming of discrete T cell subpopulations that may not be apparent using BV-BJ matrices. The sensitivity of real-time PCR for detection of variations in amounts of template can require control of cell numbers and quantitation of total RNA. The sensitivity of the methods provided herein can be based in part on the comparisons of matrices with 240 matched pairs of Ct values that provide great statistical power. Routine use of the methods provided herein to compare levels of diversity in total T cell populations can involve enrichment of T cells or CD4 and CD8 subpopulations to ensure that percentages of T cells within the populations used for RNA extractions are consistent. In some cases, amplifications of a segment of the BC region can be included in parallel with BV-BJ matrices. The Ct values from these reactions can then be used to “calibrate” Ct values from the BV-BJ matrices to minimize the effects of subtle differences in total beta transcript expression.

The BV-BJ matrices can be developed for analysis of repertoires of human T cell populations. Humans express 47 BV genes and 13 BJ genes, and these numbers can require increased attention to the design of BV-specific nested primers since the majority of BV genes are closely related members of subfamilies (Giudicelli et al., Nucl. Acids Res., 33:D256-D261 (2005)). The resulting matrices of 611 individual BV-BJ combinations can provide even greater resolution than the mouse matrices and increase the efficiency of identifying and sequencing beta transcripts from sites of T cell infiltration. BV-BJ matrices can accelerate the analyses of T cell repertoires in humans and animals through their technical simplicity, uncomplicated statistical analysis, and increased levels of resolution.

The invention will be further described in the following examples, which do not limit the scope of the invention described in the claims.

### EXAMPLES

### Example 1

### Analysis of Repertoires of T Cell Receptors

### Mice

C57B1/10SnJ (B10), C57BL/6J (B6), B10.129-H4^{b} (21M), and NOD.CB17-Prkdc^{scid}/J (NOD-scid) mice were purchased from the Jackson Laboratory (Bar Harbor, Me.). All mice were housed in the barrier facility, and all mice were raised and maintained with protocols approved an animal care and use committee.

### Cell Harvests and Skin Grafting

Lymphocyte populations were suspended by pressing spleens through nylon bolting cloth (100 μm pore size); lymphocytes were re-suspended in lysis buffer (RNeasy Protect MiniKit, Qiagen, Valencia, Calif.) for storage at −80° C. Transplantation of orthotopic tail skin grafts (about 2 mm×5 mm in size) was performed using techniques similar to those described elsewhere (Bailey and Usama, Transplantation Bulletin, 7: 424-428 (1960)). All skin grafting was performed with donors and recipients that were anesthetized with sodium pentobarbital. Each recipient of primary allografts to be scored for times of rejection received a single autograft and two allografts. Primary skin grafts were scored at routine intervals for the condition of epidermal scale pattern, pigment, and hair, and rejection was scored when no viable signs were observed for both allografts. Second sets of two skin allografts were transplanted about 14 days after rejection of the primary allografts. When the rejection process was observed on the basis of edema and ulceration, the allografts were harvested and replaced by syngeneic grafts to promote wound healing. Five cycles of grafting and harvesting were performed with each recipient with about 14 day intervals between allograft harvests and subsequent transplantation of allografts. Harvested grafts were immediately transferred to lysis buffer for storage at −80° C.

Beta Transcript Amplification. Murine TcR beta transcript repertoires include transcripts that result from rearrangements between 21 BV and 12 BJ gene segments. The following method involves the simultaneous amplification of 240 BV-BJ combinations by real-time PCR using 20 BV- and 12 BJ-specific primers (FIG. 1). Briefly, beta transcripts were first reverse-transcribed from total RNA with a biotinylated BC region reverse primer and amplified with pools of BV-specific forward primers. The resulting amplicons were mixed with streptavidin-coated magnetic beads to enrich products that include the biotinylated BC region primer. The bead-enriched products were delivered to microtiter wells for amplification in real-time PCR using 240 nested BV-BJ primer pairs.

Extraction of Total RNA

Total RNA was extracted from suspended splenocytes and tail skin grafts from individual mice using an RNeasy Protect MiniKit (Qiagen) according to the manufacturer's instructions. About 0.6 μg and 1.5-5.0 μg total RNA were extracted per million splenocytes and two skin grafts, respectively. Residual genomic DNA was removed from extracted RNA samples using an RNase-Free DNase Set (Qiagen). Total RNA was diluted to 5 ng/μL in water immediately prior to use in RT-PCRs.

### Primers

Primers were synthesized by the Invitrogen (Carlsbad, Calif.) SupplyCenter located at the Mayo Clinic Primer Core Facility (Rochester, Minn.). Sequences of 21 forward, outer primers were homologous to sequences within the CDR1 regions of BV genes (Table 1). These primers were divided into four primer pools (listed in Table 1) for use in RT-PCRs with a biotinylated beta constant region primer. Twenty nested BV primers were based on sequences within the beta CDR2 regions, and each was paired with one of 12 BJ-specific primers to create a matrix of 240 fully-nested real time PCR reactions.

```
TABLE 1
  Sequences of oligonucleotide primers used for
  amplifications in RT-PCRs and real-time PCRs.
```

| | | SEQ |
|---|---|---|
| | | ID |
| | RT-PCR Primers | NO: |
| Constant Region | Bio-GCAATCTCTGCTTTTGATGGCT | 1 |
| Reverse Primer: | (Biotinylated at 5′-end) | |
| Pool #1: | | |
| BV1 | TATGTCTTGTGGAAACAGCACTC | 2 |
| BV2 | ATGGCTTCTGTGGCTACAGACC | 3 |
| BV5.1 | AACACTGCCTTCCCTGACCC | 4 |
| BV5.2 | GTCTAACACTGTCCTCGCTGATTC | 5 |
| BV8.3 | GAAAGGTGACATTGAGCTGTCAC | 6 |
| Pool #2: | | |
| BV4 | GAAAAAATCCTGATATGCGAACAGTA | 7 |
| BV6 | CAAAAACTGACCTTGAAATGTCAA | 8 |
| BV7 | AGAATGTTTTGCTGGAATGTGGA | 9 |
| BV11 | TGCTTCTTGAGAGCAGAACCAA | 10 |
| BV12 | CAATAATCCTGAAGTGTGAGCCAG | 11 |
| Pool #3: | | |
| BV3 | AAGGACAAAAAGCAAAG ATGAGG | 12 |
| BV14 | CCTGGGCATGTTCTTGGG | 13 |
| BV15 | TATTACTTCTGGGGCCTG | 14 |
| BV16 | GTTGGATAATTTTTAGTTTCTTGGAAG | 15 |
| BV20 | GGCCAGGAAGCAGAGATGAAA | 16 |
| Pool #4: | | |
| BV8.1 | GAAAGGTGACATTGAGCTGTCAC | 17 |
| BV8.2 | GGAAAGGTGACATTGAGCTGTAAT | 18 |
| BV9 | CTTCTGTCTTCTTGCAGCCACTT | 19 |
| BV10 | TGCCTCTTGGGAATAGGCC | 20 |
| BV13 | AGTGTTCTGTCTCCTTGACACAGTAC | 21 |
| BV18 | CCTGCTACTTCTTTGGAGCCA | 22 |

Real-Time PCR Primers

| | | |
|---|---|---|
| Nested BV Primers: | | |
| BV1 | GCCCAGTCGTTTTATACCTGAAT | 23 |
| BV2 | GTGCTGATTACCTGGCCACAC | 24 |
| BV3 | GAAAAACGATTCTCTGCTGAGTGT | 25 |
| BV4 | CTTATGGACAATCAGACTGCCTCA | 26 |
| BV5.1 | ATGGAGAGAGATAAAGGAAACCTG | 27 |
| BV5.2 | GTGGAGAGAGACAAAGGATTCCTA | 28 |
| BV6 | GGCGATCTATCTGAAGGCTATGA | 29 |
| BV7 | AAGGAGACATCCCTAAAGGATACAG | 30 |
| BV8.1 + 2 | CAAGGCCTCCAGACCAAGC | 31 |
| BV8.3 | ACAAGGCCACCAGAACAACG | 32 |
| BV9 | TTCTACTATGATAAGATTTTGAACAG | 33 |
| | GG | |
| BV10 | GGCGCTTCTCACCTCAGTCTT | 34 |
| BV11 | AGATGATTCAGGGATGCCCA | 35 |
| BV12 | CAAGTCTCTTATGGAAGATGGTGG | 36 |
| BV13 | GATGAGGCTGTTATAGATAATTCACA | 37 |
| | GT | |
| BV14 | CAGGTAGAGTCGGTGGTGCAA | 38 |
| BV15 | CAGGAAAAATTTCCCATCAGTCAT | 39 |
| BV16 | GGAGAAGTCTAAACTGTTTAAGGATC | 40 |
| | AG | |
| BV18 | AAGGACAAGTTTCCAATCAGCC | 41 |
| BJ primers: | | |
| BJ1.1 | CTGGTTCCTTTACCAAAGAAGACT | 42 |
| BJ1.2 | CCCTGAGCCGAAGGTGTAGTC | 43 |
| BJ1.3 | TTCTCCAAAATAGAGCGTATTTCC | 44 |
| BJ1.4 | GGTTCCATGACCGAAAAATAATCT | 45 |
| BJ1.5 | CTCCAAAAAGCGGAGCCTG | 46 |
| BJ1.6 | CGCAAAGTAGAGGGGCGAA | 47 |
| BJ2.1 | CTGGTCCGAAGAACTGCTCA | 48 |
| BJ2.2 | CCAAAGTAGAGCTGCCCGGT | 49 |
| BJ2.3 | CCTGAGCCAAAATACAGCGTT | 50 |
| BJ2.4 | GCACCAAAGTACAAGGTGTTTTG | 51 |
| BJ2.5 | GGCCCAAAGTACTGGGTGTC | 52 |
| BJ2.7 | GCCGGGACCGAAGTACTGT | 53 |

### RT-PCRs

Four pooled RT-PCRs were performed in 50 μL volumes using a One-Step RT-PCR Kit (Qiagen), 15 ng of total RNA, 20 pmol of a 5′-biotinylated BC primer, and pools of BV primers (three pools of five primers and one pool of six primers) that provided 6.6 pmol of each BV primer. RNA templates were denatured at 75° C. for 4 minutes and placed on ice prior to addition to RT-PCR reactions. Cycling was performed on a PTC-225 Peltier Thermal Cycler (MJ Research, Waltham, Mass.) as follows. cDNA synthesis was performed at 50° C. for 32 minutes followed by incubation at 95° C. for 15 minutes to inactivate the reverse transcriptase. Subsequent PCR parameters were 1 minute at 94° C., 30 seconds at 60° C., and 1 minute at 72° C. for 25 cycles. A final extension cycle was performed for 6 minutes at 72° C. RT-PCR products were separated from residual primers and amplification reagents using a QIAquick PCR Purification Kit (Qiagen) and eluted with 50 μL of elution buffer.

### Enrichment of Biotinylated PCR Products

Biotinylated RT-PCR products were purified with My One™ Streptavidin C1 Dynabeads (Dynal Biotech ASA, Oslo, Norway) following the manufacturer's protocol. Briefly, 50 μL of Dynabeads were washed two times in 50 μL of 2× washing and binding buffer (10 mM Tris-HCl (pH 7.5), 1 mM EDTA, and 2 M NaCl). Following the second wash, the beads were resuspended in 100 μL of 2× washing and binding buffer, 50 μL of PCR product, and 50 μL of sterile water. The suspensions were incubated for 15 minutes at room temperature with gentle shaking. The amplicon-bound beads were washed twice with 100 μL, of 1× washing and binding buffer and then resuspended in 100 μL of 10 mM Tris-HCl, pH 8.5. Suspensions of amplicon-bound beads were diluted 1:10 for direct use as templates in real time PCR reactions.

### Real Time PCR

A total of 240 individual real-time PCRs (20 BV and 12 BJ primers) were performed in 10 μL volumes in 384-well Clear Optical Reaction Plates with Optical Adhesive Covers (Applied Biosystems, Foster City, Calif.). The components of reactions were 10 pmol of a nested BV primer (Table 1), 10 pmol of a BJ-specific primer (Table 1), 10 μL of the respective amplicon-bound bead suspension, and 50 μL Power SYBR Green PCR Master Mix (2×) (Applied Biosystems). Cycling was performed on an ABI Prism 7900HT Sequence Detection System at the AGTC Microarray Shared Resource Core Facility (Mayo Clinic) using SYBR Green detection. Cycling parameters were as follows: (1) an initial incubation at 50° C. for 2 minutes, (2) a 10 minute incubation at 95° C. to activate the DNA polymerase, and (3) 40 cycles of 15 seconds at 95° C. followed by 1 minute at 60° C. Dissociation curves were generated by (1) incubating the amplicons at 95° C. for 15 seconds, (2) reducing the temperature to 60° for 15 seconds, and (3) increasing the temperature to 95° C. over a dissociation time of 20 minutes. Data were analyzed with the 7900HT SequenceDetectionSystem (SDS) Version 2.3 software (Applied Biosystems) to estimate cycle threshold (Ct) values and dissociation curves to estimate the optimal melting temperatures for all reactions. Ct values are fractional cycle numbers at which fluorescence passes the threshold level (designated by a horizontal line in Ct plots), that is automatically set to be within the exponential region of the amplification curve where there is a linear relationship between the log of change in fluorescence and cycle number. Dissociation curves are formed by plotting rising temperatures versus the change in fluorescence/change in temperature.

### Sequence Analysis

Real-time PCR products were cleaned using a QIAquick PCR Purification Kit (Qiagen) prior to sequencing with 2 pmol of the respective, nested BV primers. Sequencing was performed by the Mayo Clinic Molecular Biology Core Facility using a Big Dye Terminator v1.1 Cycle Sequencing Kit (Applied Biosystems) prior to analysis of all sequences on a 96-capillary ABI PRISM™ 3730 XL DNA Analyzer (Applied Biosystems) by the Mayo Clinic Molecular Biology Core Facility.

### Statistical Analysis

Wilcoxon matched pairs and Kruskal-Wallis tests were used to estimate the statistical significance of differences in representation of BV-BJ combinations. The relative abundance of BV-BJ combinations was defined by the observed Ct values and dissociation curves. Dissociation curves were used to confirm the presence of amplicons from beta transcripts by excluding (1) primer-dimers that had relatively low melting temperatures and (2) amplicons with peak heights that did not exceed a threshold of 0.07 (change in fluorescence/change in temperature). This threshold was selected due to the inability to sequence amplicons that were below this value. Amplicons with either or both of these characteristics were assigned Ct values of >40 cycles. Arrays of Ct values were paired according to BV-BJ combinations and Wilcoxon matched pairs and Kruskal-Wallis tests were performed with Prism software (GraphPad Software, San Diego, Calif.).

The diversities of the 240 BV-BJ combinations within individual RNA templates also were estimated by Shannon entropy (Shannon, The Bell System Technical Journal, 27:379-423 & 623-656 (1948)) that has been used for estimating variability at individual amino acid positions in immunoglobulin variable region gene products (Litwin and Jores, In Perelson and Weisbuch (ed.) Theoretical and experimental insights into immunology, Springer-Verlag, Berlin (1992)). An estimate of scaled entropy (H) was calculated for each BV-BJ matrix by the equation H=Σ (p log 2 p)/log 2 (1/240) where p was the probability of abundance calculated for each BV-BJ combination by the equation p=2−y/Σ2−y where y was the Ct value for the BV-BJ primer pair and p=0 when Ct>40 cycles. Scaled entropy ranges from zero to one with one representing maximal diversity.

### Results

The diversity of expressed combinations of individual BV and BJ genes is a major contributor to the diversity of TcR repertoires. Based on 21 BV and 12 BJ genes, 252 BV-BJ combinations can be expressed in mice. The relatively large BV and BJ gene families can provide an approach to analyze beta transcript repertoires with increased resolution. The homologies within the BV and BJ gene families can require selection of primers to ensure specific amplification of transcripts carrying individual BV-BJ combinations.

All primers were designed with comparable Tm's of approximately 60° C. In general, the BV-specific primers were homologous to sequences within the CDR1 (outer primers) and CDR2 (nested primers) regions. Twenty pairs of nested, forward primers were designed to amplify the 21 expressed BV genes. Choices for optimization of outer primers for the CDR1 regions of BV8.1 and BV8.2 in the CDR1 region were accomplished by designing individual primers for these two genes. However, a single primer was selected for the BV8.1 and BV8.2 genes within the CDR2 region since they could not be separately amplified at the nested stage under conditions required for the other BV-specific primers. BJ-specific primers were designed for each of the 12 expressed BJ genes.

The flow of the experimental method is presented in FIG. 1. Templates for real-time PCRs were amplified in RT-PCR's using (1) total RNA as template, (2) a reverse constant region primer that was biotinylated at the 5′ end, and (3) four pools of five to six BV-specific primers. BV-specific primers were placed in pools on the basis of relative homology to minimize cross-priming. Amplified products were cleaned by direct column purification to remove excess primers prior to mixing with streptavidin-coated magnetic beads to enrich products that were specifically amplified from beta transcripts by washing away non-specific amplicons. Amplicon-bound beads were aliquoted into the wells of 384-well plates along with single nested BV-specific primers and single BJ primers. Amplification was monitored by the uptake of SYBR Green with automated estimation of Ct values throughout each reaction. Dissociation curves were generated after the final amplification cycle by increasing the temperature from 60° C. to 95° C.

Repertoires in Normal Mice

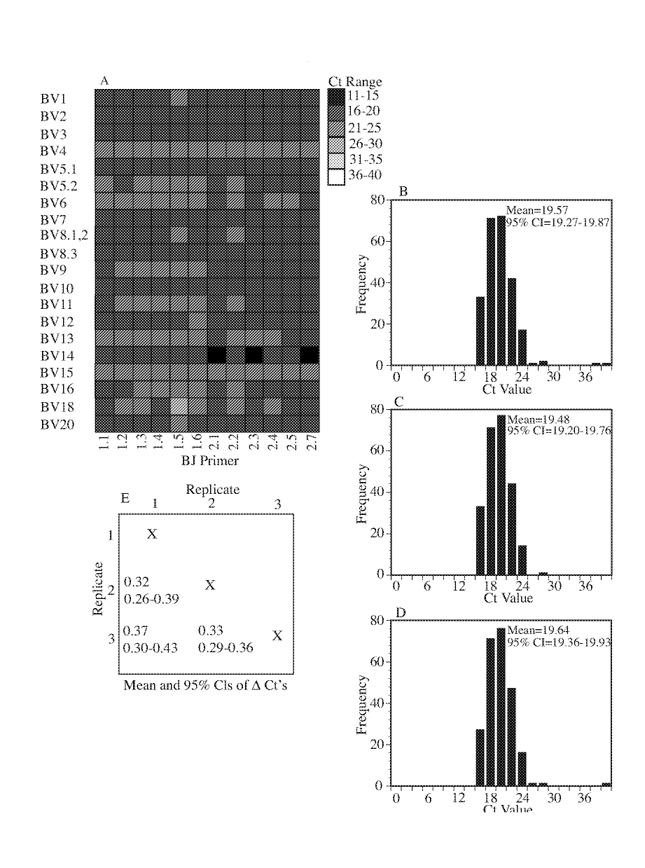

BV-BJ matrices were first used for the analysis of beta transcript repertoires in lymphocyte populations from normal mice. Total RNA was extracted from splenocytes collected from one normal B6 mouse and one normal B10 mouse, and 15 ng RNA/pool were amplified in each of four pooled RT-PCRs to generate templates for real-time PCRs performed with individual BV-BJ primer pairs. Ct values were estimated for each BV-BJ combination, and the vast majority (95% and 94%) were between 16 and 25 cycles (FIGS. 10A and B). These results demonstrate that all 240 BV-BJ combinations can be amplified with the utilized primer sets.

Amplification in a total of 25 RT-PCR cycles and 40 real-time PCR cycles requires attention to potential sources of experimental error. Two additional replicate assays were performed with the same source of RNA template to evaluate reproducibility (FIGS. 10C-D). The mean Ct values for the replicate matrices (19.57 and 19.64) were virtually identical to that of the original matrix (19.48 cycles) with comparable distributions. The replicate distributions of Ct values were also compared by calculating (1) the delta Ct (ΔCt) values between individual, replicate BV-BJ combinations and (2) the mean ΔCts for all three comparisons of replicate matrices. The mean ΔCts ranged from 0.32-0.37 (FIG. 10E), and these ranges were well within the sensitivity range of ±0.5 cycles reported with the use of gene expression master mixes for real-time PCR according to Applied Biosystems. The effects of different lymphocyte sources of total RNA on the reproducibility of results from BV-BJ matrices can be assessed by comparing the mean Ct values, described in FIGS. 10-13, that were obtained with RNA extracted from splenocytes harvested from multiple C57 background mice.

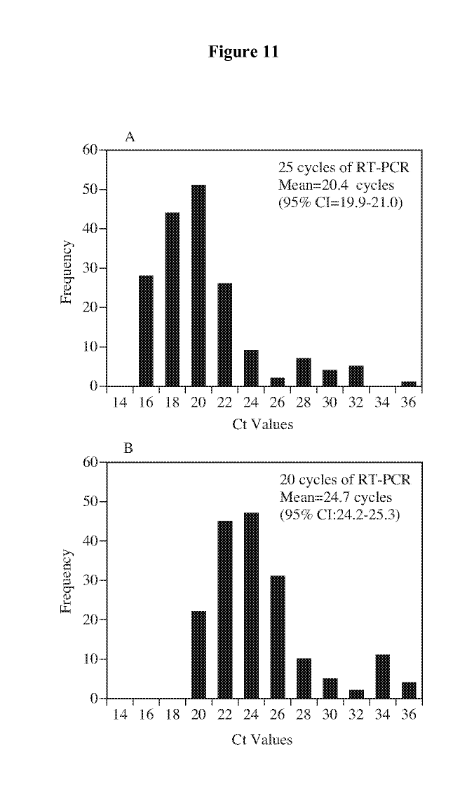

The amplification of beta transcripts with outer BV primers in RT-PCRs increased the specificity of amplifications in fully nested BV-BJ-specific real-time PCRs. However, RT-PCR amplifications through 25 cycles could potentially lead to saturated product levels, which could distort the distributions of beta transcript products and, therefore, alter the results of the BV-BJ matrix. The effects of reducing the number of RT-PCR cycles on mean Ct values from BV-BJ primer pairs in real-time PCRs was investigated. RNA template (15 ng) from normal B6 splenocytes was amplified for 20 and 25 cycles in RT-PCRs. Amplified products were bead-enriched and amplified with 180 BV-BJ primer pairs in real-time PCRs. The reduction of the RT-PCRs to 20 cycles resulted in an increase in mean Ct value of 4.3 cycles (FIG. 11). These results suggested that the accumulation of products in the RT-PCRs was not saturated over the 20-25 cycle range.

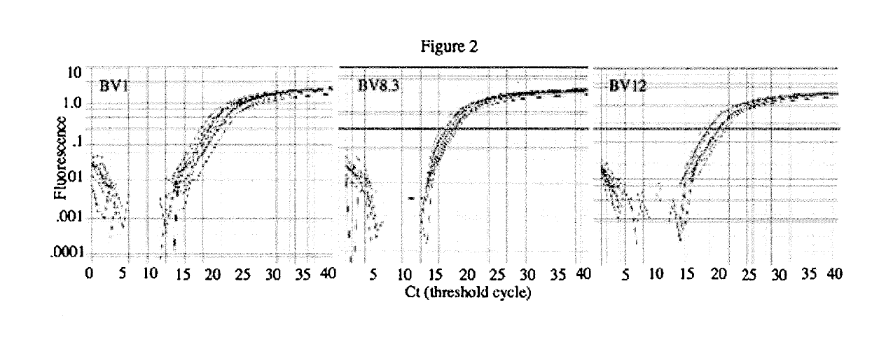

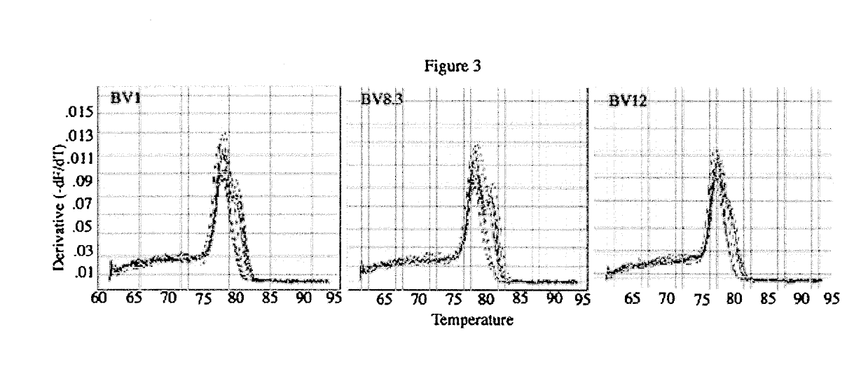

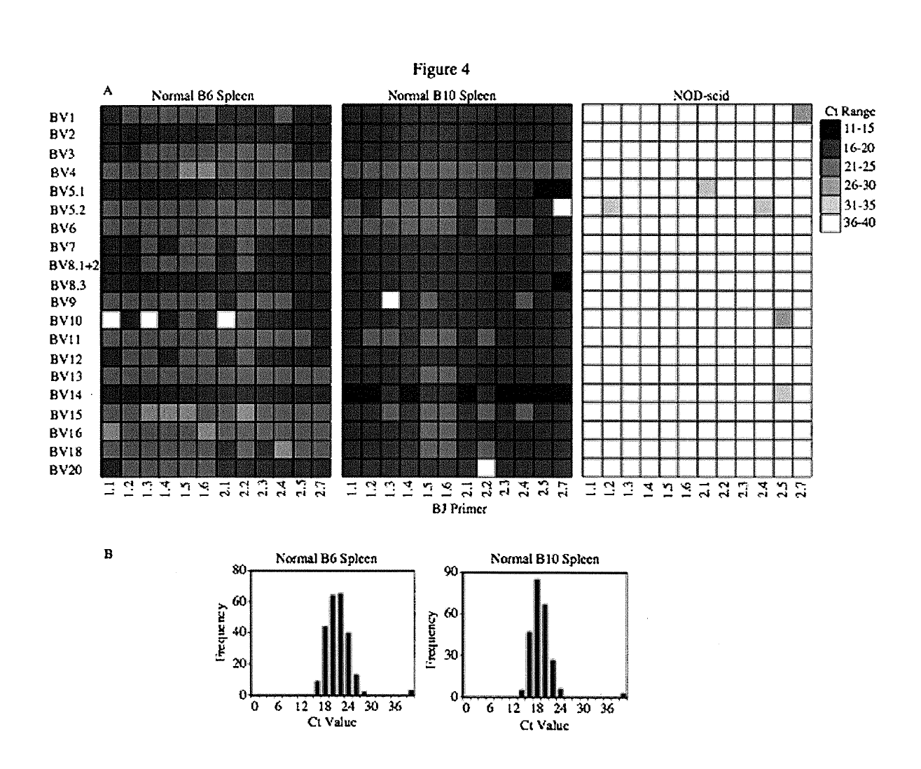

The tempos of amplification of different BV-BJ combinations appeared to be comparable (FIG. 2 for representative combinations). Dissociation curves were inspected to confirm the amplification of beta transcripts and exclude primer-dimers based on the curves of melting temperatures. Virtually all (99%) BV-BJ combinations exhibited melting curves consistent with amplification of beta transcripts (FIG. 3 for representative dissociation curves). Lack of specific amplification was concluded if there had been either a low melting temperature, indicative of primer-dimer, or SYBR Green uptake had not reached the threshold for estimation of a Ct value. In those cases, Ct values of >40 cycles were assigned. Taking the Ct values and confirmation of specific amplification with dissociation curves into consideration, the results of this analysis of normal splenocytes were summarized (FIG. 4). Virtually all BV-BJ combinations were amplified with lack of specific amplification with only 3/240 BV-BJ combinations. These collective results demonstrate that all 240 BV-BJ combinations can be amplified with the utilized primer sets. Two additional RNA templates, that were extracted from splenocytes from immunodeficient NOD-scid mice (T and B cell-deficient), were amplified by BV-BJ matrices (FIG. 1 for one template). Amplification was observed with only 6/240 BV-BJ primer pairs, and the two products with Ct values between 26 and 30 were sequenced and confirmed to be single TcR beta transcripts. These results demonstrate that BV-BJ matrices specifically amplify TcR beta transcripts.

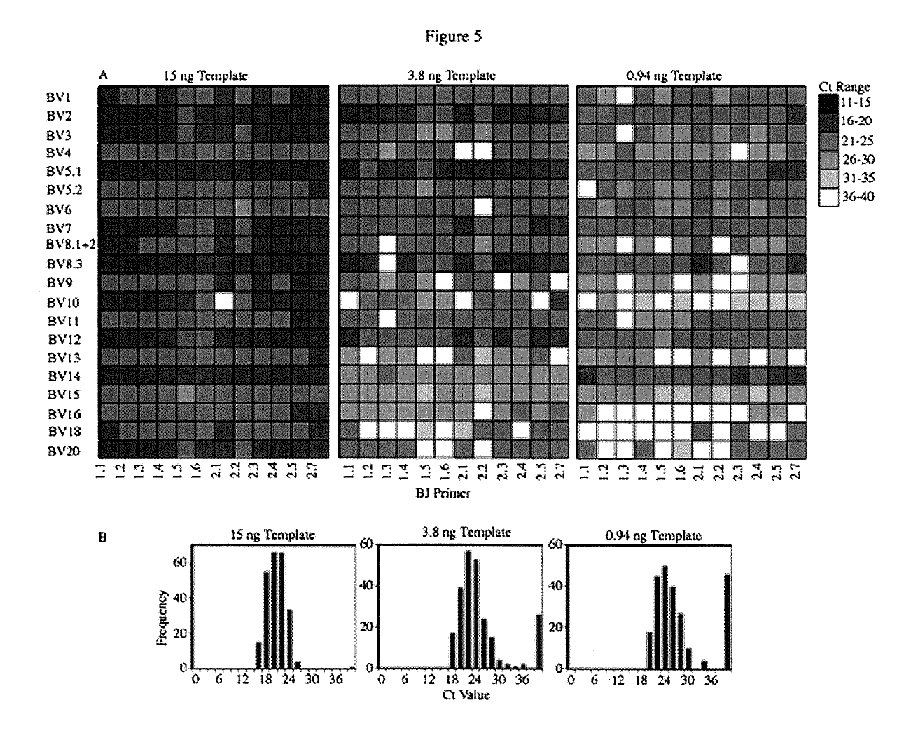

BV genes can be differentially expressed in normal T cell populations (Robinson, Hum. Immunol., 35:60-67 (1992); Vacchio and Hodes, J. Exp. Med., 170:1335-1346 (1989); and Pullen et al., J. Exp. Med., 171:49-62 (1990)), and data from a more limited number of experiments demonstrate that BJ genes can be differentially expressed by T cell subpopulations expressing single BV genes (Feeney, J. Exp. Med., 174:115-124 (1991) and Candeias et al., J. Exp. Med., 174:989-1000 (1991)). If individual BV-BJ combinations are variably expressed in T cell populations, then amplifications of these BV-BJ pairings in real-time PCR should be variably affected by amounts of RNA template. The selection of 15 ng RNA template/pooled RT-PCR was based on the observations that this amount of template dependably yielded amplification for all BV-BJ combinations. Therefore, two additional analyses were performed using RNA template diluted ¼ (3.75 ng/pool) and 1/16 (0.94 ng/pool). The speed of amplification as well as detection of products were strongly dependent on amounts of RNA template (FIG. 5). Increasing reductions in template resulted in trends toward increased Ct values, and percentages of BV-BJ combinations for which no amplification was detected. The dissection of repertoires into matrices of defined BV-BJ combinations facilitated statistical analysis using methods designed for comparing two or more groups of paired data. The statistical significance of the effects of template titration was estimated by (1) the Wilcoxon matched pairs test for comparisons of two matrices and (2) the Kruskal-Wallis test for comparisons within the group of three matrices. The Kruskal-Wallis test was followed by Dunn's post test for all pairwise comparisons of matrices. The matching of Ct values for individual BV-BJ combinations in both of these tests eliminates potential complications from natural over-representation of specific BV genes and differences in efficiencies of BV-BJ primer pairs. Observed differences in diversity associated with reduction in RNA template were significant at p<0.0001 using both forms of analysis.

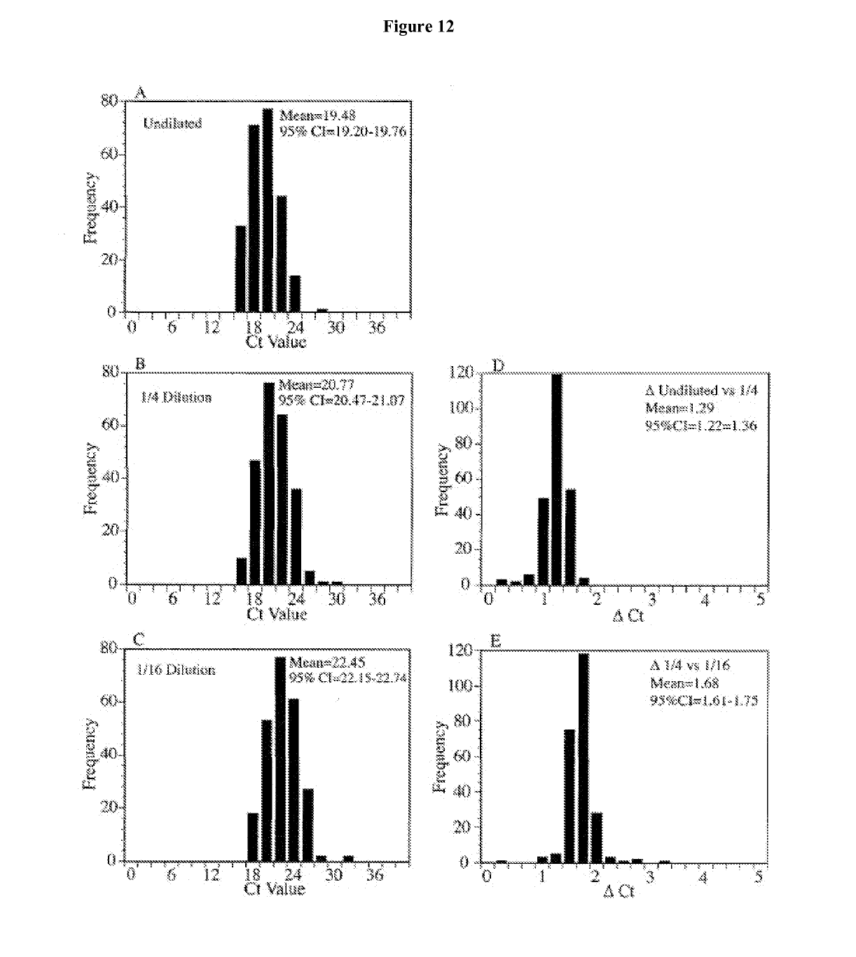

Effects of Dilutions of Templates for RT-PCRs and Real-Time PCRs. BV-BJ primer pairs with comparable efficiencies are desired for maximal detection of transcripts with variable levels of representation. Comparable primer pair efficiencies should yield comparable increases in Ct values following dilution of templates for real-time PCRs. Pooled RT-PCRs were performed, and the bead-enriched products from each pooled RT-PCR were used in real-time PCRs undiluted (as per standard protocol) and diluted ¼ and 1/16 (FIG. 12). A ¼ dilution resulted in a mean increase of 1.29 cycles, and a 1/16 dilution resulted in an additional mean increase of 1.68 cycles (FIG. 12A-C). More importantly, >90% of the BV-BJ combinations exhibited ΔCt values in the 1.0-1.75 cycle interval in the undiluted vs. ¼ comparison, and, likewise, >90% of the combinations exhibited ΔCt values in the 1.25-2.00 interval for the ¼ vs. 1/16 comparison (FIGS. 12D and E). Further, no consistent effect of titration (undiluted → 1/16) was observed for the <10% of BV-BJ combinations whose ΔCt values fell outside of these ranges suggesting that these shifts were not due to reproducible differences in BV-BJ primer pair efficiencies.

Normal T cell populations exhibit variable levels of expression of both BV and BJ genes (Vacchio and Hodes, J. Exp. Med., 170:1335-1346 (1989); Pullen et al., J. Exp. Med., 171:49-62 (1990); Candeias et al., J. Exp. Med., 174:989-1000 (1991); and Kato et al., Eur. J. Immunol., 24:2410-2414 (1994)), and it could be expected that reducing the amount of starting RNA template for the BV-BJ matrix results in the loss of detection of transcripts that carry BV-BJ combinations that are low in abundance. The data presented herein indicate that the speed of amplification in the real-time PCR phase of the BV-BJ matrix method was directly related to the amount of bead-enriched template. However, the Ct values in the BV-BJ matrix were the product of amplification in both the real-time PCRs as well as the pooled RT-PCRs. The RT-PCRs were more complex reactions given the heterogeneous template and pooled BV primers that potentially could result in non-specific amplification and competition between BV primers for amplification with the biotinylated BC primer. Accordingly, these amplifications may be more sensitive to variations in amounts of template RNA.

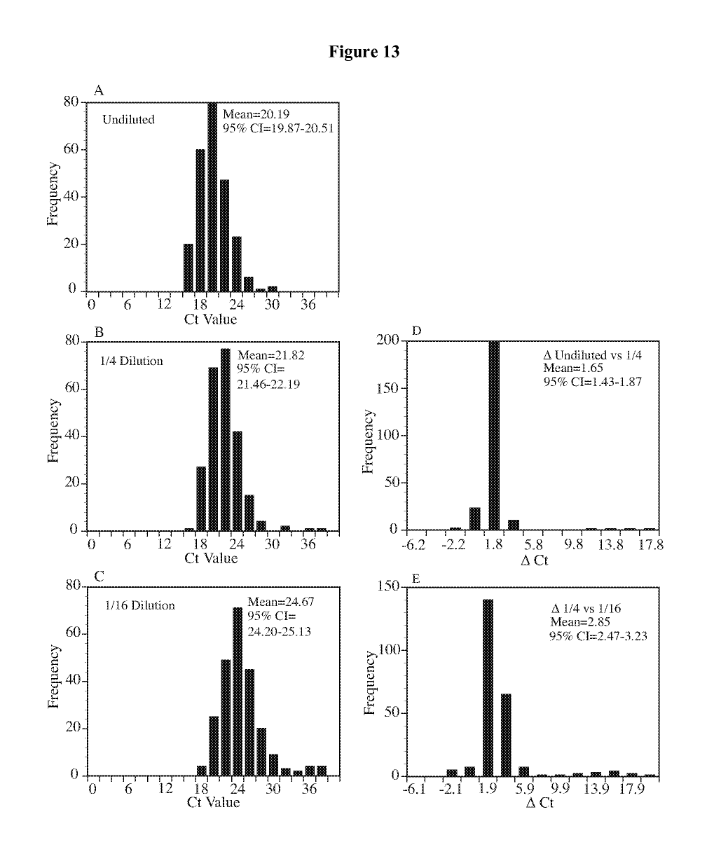

The effects of RNA titration on amplification with BV-BJ primer pairs were investigated. Total RNA was extracted from B6 splenocytes and amplified in pooled RT-PCRs after either no dilution or ¼ and 1/16 dilutions. Bead-enriched templates were then amplified in real-time PCRs to evaluate the effects of RNA template dilution on mean Ct values and ΔCt values for individual BV-BJ primer pairs. Reductions in amounts of RNA template resulted in increases in mean Ct values (FIG. 13A-C) with increased tailing toward higher Ct values with extended ( 1/16) dilution. Further, the breadths of the major peaks of ΔCt values were increased following dilution (FIGS. 13D and E) over those observed with ¼ and 1/16 dilutions of bead-enriched templates for real-time PCRs (FIGS. 12D and E). The increases in ΔCt variability were not unexpected given that template dilutions were made prior to the pooled RT-PCRs that utilize total RNA template and multiple, pooled BV primers. This increased variability extended further to a minority (<10%) of BV-BJ combinations that exhibited ΔCt values (¼ vs 1/16) that exceeded those expected for a four-fold dilution. A close examination of the Ct values for these BV-BJ combinations revealed that there was no clear correlation between the Ct values with ¼ diluted template and their ΔCt values (¼ vs. 1/16). The effects of RNA template dilution on Shannon entropy estimates of diversity were investigated, and scaled entropy values of 0.86, 0.85, and 0.81 were calculated for the undiluted, ¼ diluted, and 1/16 diluted RNA templates, respectively. The estimated loss in diversity with increasing template dilution ( 1/16) appeared to be due to a small number of BV-BJ primer pairs that yielded no amplification (Ct>40 cycles).

Considering the results of titrations of both bead-enriched template and starting RNA template, it is apparent that amplifications with the vast majority of BV-BJ primer pairs responded concordantly to template titrations. However, data in FIGS. 12 and 13 show that template can be reduced to an amount where ΔCts from a minority of BV-BJ-specific amplifications exceed the values predicted by the template dilution resulting in a loss of representation. Since there can be a wide range of representation of transcripts with different BV-BJ combinations, one should consider selecting amounts of starting RNA for RT-PCRs and bead-enriched templates for real-time PCRs that (1) exceed the amounts required for detection of highly represented transcripts and (2) are sufficient for detection of poorly represented transcripts to maximize the observable diversity of BV-BJ combinations.

Detection of Variable Diversity. The analyses of multiple inbred mice with the BV-BJ matrix revealed that BV-BJ combinations exhibit only minor variations in representation in normal C57 background mice. This relative homogeneity may be based in the housing of these genetically identical mice under specific pathogen-free conditions that do not exert significant selective pressures on T cell populations. The ability of the BV-BJ matrix to detect repertoires with reduced or skewed diversity was investigated through the use of genetically immunocompromised mice and populations of lymphocytes that were purposefully mixed with monoclonal T cells.

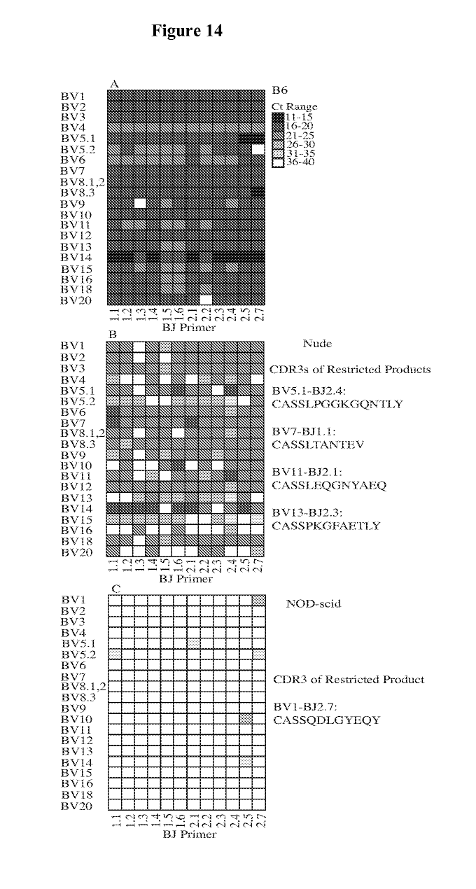

Immunocompromised mice included (1) NOD-scid mice that lack B and T cells and (2) nude mice that are athymic but capable of low levels of extra-thymic T cell development leading to accumulations of detectable T cell populations with increasing age (Kennedy et al., J. Immunol., 148:1620-1629 (1992)). Spleens were harvested from nude mice at 16 wk of age based on previous observations that populations of CD4⁺ and CD8⁺ T cells have accumulated by that age (Kennedy et al., J. Immunol., 148:1620-1629 (1992)). B cells were depleted from nude spleen cells by panning over dishes coated with goat anti-mouse Ig. The eluted cells were 50% T cells based on flow cytometric analysis using fluorochrome-labeled antibodies specific for CD3, CD8, and CD4. Total RNA was extracted from these populations and analyzed by the BV-BJ matrix (FIG. 14B for one representative mouse). The results of the BV-BJ analysis of nude T cells differed from those of normal B10 T cells (FIG. 14A) in two principal respects: (1) the median Ct value (23.9 cycles) was 5.4 cycles slower than the median Ct value for normal B10 T cells and (2) no amplification was observed for 20% of the BV-BJ combinations. The significance value for the apparent reduction in diversity of the nude TcR repertoire was estimated at p<0.0001 by the Wilcoxon matched pairs test that utilizes ΔCt values estimated for all 240 BV-BJ combinations. The apparent reduction in diversity of the nude TcR repertoire was supported by Shannon entropy analysis that yielded scaled entropy values of 0.88 and 0.73 for normal and nude T cells, respectively. Reduced diversity in the nude BV-BJ matrix was also indicated by the identification of amplicons from the real-time PCRs that had dissociation curves with reduced breadth suggesting reduced complexity. Six amplicons of this type were selected for direct sequencing and four amplicons yielded single-copy sequences for clear translations of the CDR3s (included in FIG. 14B). Greater reduction in BV-BJ diversity was observed through analysis of splenocytes from an NOD-scid mouse. NOD-scid mice lack B and T cells due to a mutation in the Prkdc gene (Bosma et al., Curr. Top. Microbiol. Immunol., 137:197-202 (1988)) so they provide a physiological negative control for the specificities of the BV and BJ primers. Total RNA extracted from whole splenocyte populations were amplified with the BV-BJ matrix method (FIG. 14C for one sample). Products were obtained for only six BV-BJ combinations with Ct values ranging from 26.4 to 39.2 cycles. At least one of these products derived from a single transcript as evidenced by the ability to obtain single copy sequence (included in FIG. 14C). These six BV-BJ primer pairs did not yield products with the second RNA template. These results indicated that the amplifications in the BV-BJ matrix method required RNA extracted from T lymphocytes.

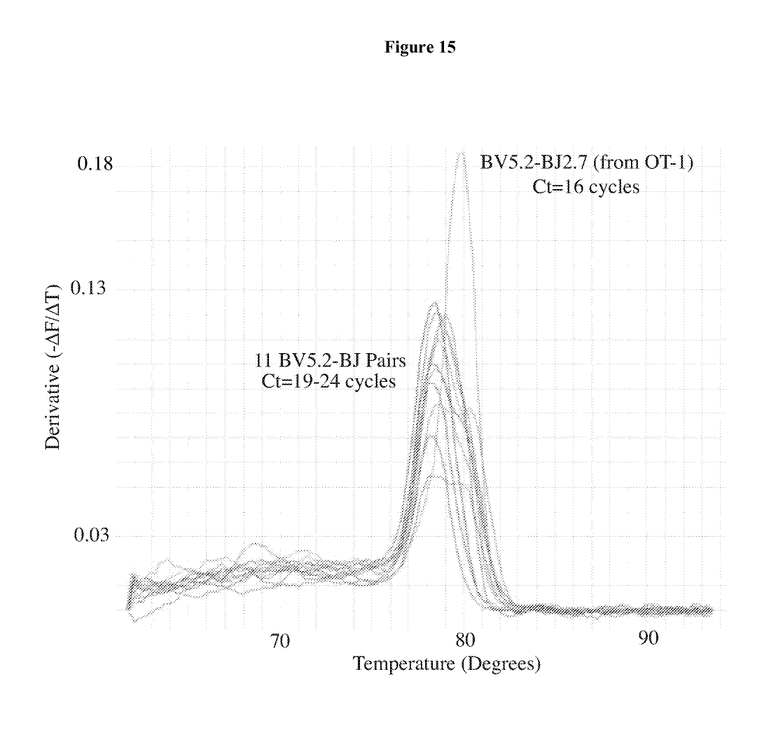

The identification of single copy sequences in amplicons derived from nude mouse T cells suggests that the BV-BJ matrix method is capable of amplifying and identifying over-represented transcripts for direct sequencing. An additional test involved the mixing of normal T cell populations with limited numbers of monoclonal T cells prior to total RNA extraction. Normal B6 spleen cells were mixed in a 100:1 ratio with splenocytes from an OT-1 transgenic mouse whose T cells expressed a BV5.2-BJ2.7 rearrangement (Hogquist et al., Cell, 76:17-27 (1994)). Total RNA that was extracted from the mixed cells was amplified in pooled RT-PCRs and re-amplified by real-time PCRs. The dissociation curves for wells combining the BV5.2 primer with the 12 BJ primers are presented in FIG. 15. The BV5.2-BJ2.7 primer pair yielded accelerated amplification (Ct=16 cycles) when compared with Ct values from the other 11 BV5.2-BJ primer pairs (19-24 cycles). Further, the dissociation peak from the BV5.2-BJ2.7 primer pair had increased magnitude and reduced width. This product was directly sequenced and yielded single-copy sequence which translated into the reported OT-1 CDR3 sequence (CASSRANYEQY (SEQ ID NO:160)). These results and those presented herein for amplification of RNA from nude mice demonstrate that the BV-BJ matrix has the capacity to identify and ultimately sequence over-represented TcR beta transcripts within whole populations of lymphocytes.

### Restricted Repertoires at Inflammatory Sites

The separate amplifications of 240 individual BV-BJ combinations can increase the capacity for identifying and sequencing amplicons from beta transcripts expressed by T cell populations that infiltrate sites of inflammation. The ability of the BV-BJ matrices to identify over-represented transcripts was investigated using a model of skin allograft rejection. Successive sets of skin allografts that are incompatible for a single minor histocompatibility antigen (MiHA) were infiltrated by changing populations of T cells (Wettstein et al., Intl. Immunol., 19:523-534 (2007)). These experiments included spectratyping to identify beta and alpha transcripts that were over-represented at the time of allograft rejection. As described herein, fifth set allografts that expressed either the H4 or HY MiHAs were harvested and were in the process of being rejected. Total RNA was extracted from the rejecting allografts and amplified in pooled RT-PCRs and subsequent real-time PCRs.

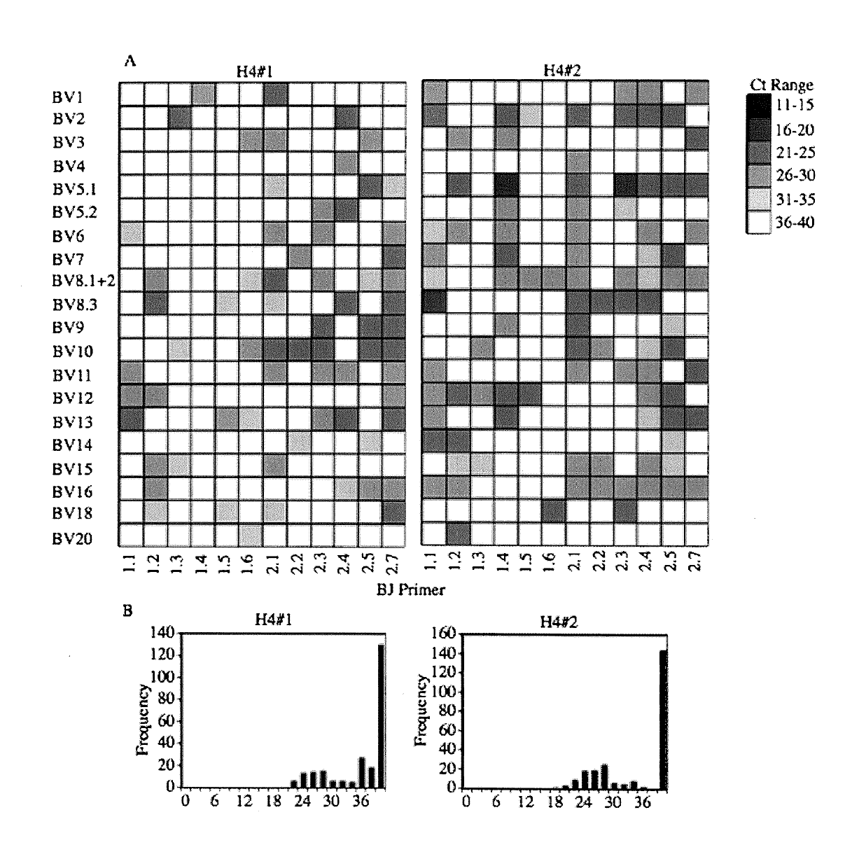

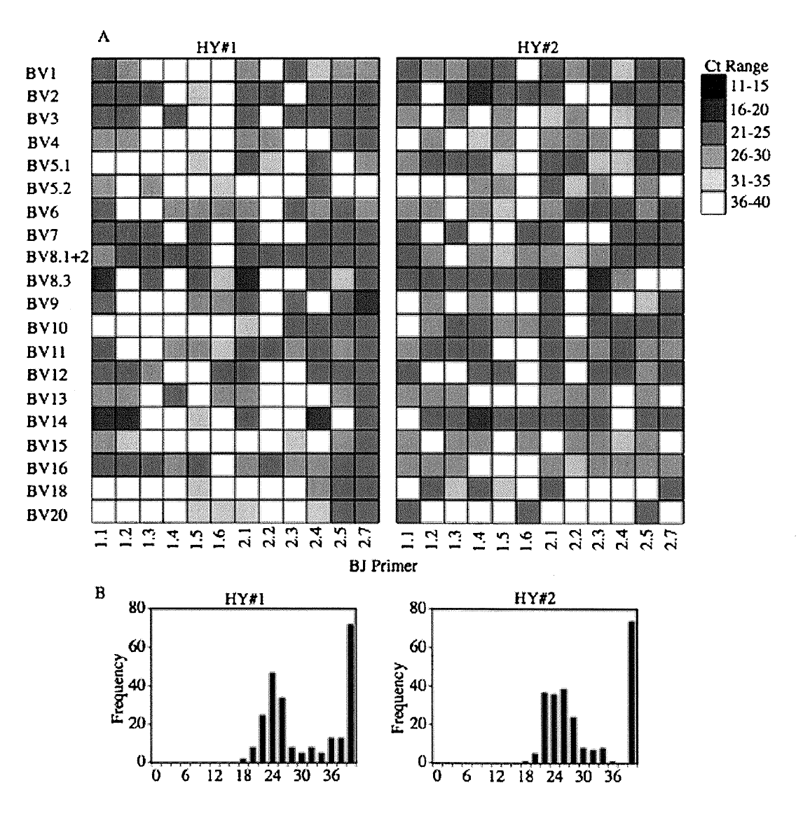

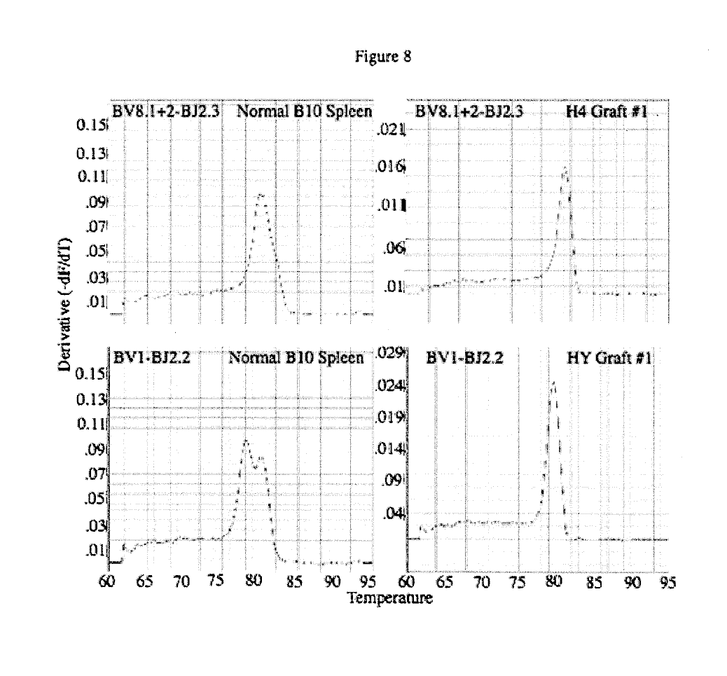

The matrices from allografts harvested from two recipients for each MiHA demonstrate significantly reduced diversity in comparison to matrices from normal T cell populations (FIGS. 6 and 7). Specific amplification was observed for only 28-40% (H4) and 60-70% (HY) of the BV-BJ combinations. Ct values were increased over those observed with normal T cells indicating reduced amounts of beta transcript templates. In addition, the widths of dissociation curves were highly variable in comparison to the same amplifications of templates from normal mice (FIG. 8) suggesting that the amplified products were variable in their complexity. Working under the assumption that the most narrow dissociation peaks included single products, sets of BV-BJ products were selected from the matrices from single recipients for sequencing with the respective BV primers. Single copy sequences with productive rearrangements were obtained for 81% of the sequenced BV-BJ products from both H4 and HY-incompatible grafts (Tables 2 and 3). All sequences included the expected BV sequences indicating that the BV-BJ amplifications were specific. Multiple products were observed with 13% and 12% of the products from H4- and HY-incompatible grafts, respectively, and 5% (H4) and 6% (HY) of the products were non-productive rearrangements. Only 1% of the products could not be sequenced due to low amounts of product. The CDR3s that were derived from the H4-incompatible grafts were inspected for net charge and length to compare them to CDR3 sequences that were previously obtained from multiple sets of H4-incompatible grafts (Wettstein et al., Intl. Immunol., 19:523-534 (2007)). The mean net charges (−0.8) and lengths (8.4 a.a.) were comparable to the CDR3s previously obtained from fifth graft sets.

TABLE 2
Amino acid sequences of beta CDR3s
over-represented in H4-incompatible
skin grafts at the time of rejection
by a single recipient (H4 #1).

| | | SEQ ID |
|---|---|---|
| BV-BJ Pair | BV/CDR3/BJ Sequence | NO: |
| BV1-BJ2.3 | CASS/LDWGG/AETLYFGSGTRLTVL | 54 |
| BV1-BJ2.4 | CASS/SQDWG/QNTLYFGAGTRLSVL | 55 |
| BV1-BJ2.7 | CASS/SDRV/QYFGPGTRLTVL | 56 |
| BV2-BJ1.1 | CSA/DRPGAS/TEVFFGKGTRLTVV | 57 |
| BV2-BJ1.4 | CSA/GTT/NERLFFGHGTKLSVL | 58 |
| BV2-BJ2.1 | CS/V/NYAEQFFGPGTRLTVL | 59 |
| BV2-BJ2.5 | CSA/DCS/QDTQYFGPGTRLLVL | 60 |
| BV3-BJ1.2 | CASS/RT/NSDYTFGSGTRLLVI | 61 |
| BV3-BJ1.4 | CAG/TGGP/NERLFFGHGTKLSVL | 62 |
| BV3-BJ2.1 | CASS/LSGR/AEQFFGPGTRLTVL | 63 |
| BV3-BJ2.7 | CASS/LGDD/EQYFGPGTRLTVL | 64 |
| BV5.1-BJ1.2 | CASS/QG/NSDYTFGSGTRLLVI | 65 |
| BV5.1-BJ1.4 | CASS/LERG/SNERLFFGHGTKLSV | 66 |
| | L | |
| BV5.1-BJ2.1 | CASS/PGLG/YAEQFFGPGTRLTVL | 67 |
| BV5.1-BJ2.4 | CASS/LALGG/SQNTLYFGAGTRLS | 68 |
| | VL | |
| BV5.1-BJ2.7 | CASS/LAGGG/YEQQFGPGTRLTVL | 69 |
| BV6-BJ1.1 | CASS/FGQGA/EVFFGKGTRLTVV | 70 |
| BV6-BJ1.2 | CASS/IRD/SDYTFGSGTRLLVI | 71 |
| BV6-BJ2.1 | CASS/IRD/NYAEQFFGPGTRLTVL | 72 |
| BV7-BJ1.1 | CASP/QVA/NTEVFFGKGTRLTVV | 73 |
| BV7-BJ2.5 | CASS/PGQG/DTQYFGPGTRLLVL | 74 |
| BV8.1-BJ2.1 | CASS/DQGD/YAEQFFGPGTRLTVL | 75 |
| BV8.1-BJ2.5 | CASS/GTG/QDTQYFGPGTRLLVL | 76 |
| BV8.2-BJ1.5 | CASG/VQGG/NQAPLFGEGTRLSVL | 77 |
| BV8.2-BJ2.3 | CASG/DLGG/SAETLYFGSGTRLT | 78 |
| | VL | |
| BV8.3-BJ1.1 | CASS/DGTV/EVFFGKGTRLTVV | 79 |
| BV8.3-BJ1.2 | CASR/GPA/NSDYTFGSGTRLLVI | 80 |
| BV8.3-BJ2.2 | CASS/E/NTGQLYFGEGSKLTVL | 81 |
| BV8.3-BJ2.4 | CASS/DWGF/QNTLYFGAGTRLSVL | 82 |
| BV9-BJ2.5 | CASS/RDTGAR/DTQYFGPGTRLLVL | 83 |
| BV10-BJ1.3 | CASS/GRS/SGNTLYFGEGSRLIVV | 84 |
| BV10-BJ2.2 | CASS/LDWR/NTGQLYFGEGSKLTVL | 85 |
| BV11-BJ1.1 | CAS S/LGNA/NTEVFFGKGTRLTVV | 86 |
| BV11-BJ1.3 | CASS/LGT/SGNTLYFGEGSRLIVV | 87 |
| BV11-BJ2.3 | CAS S/PGTG/AETLYFGSGTRLTVL | 88 |
| BV11-BJ2.4 | CASS/LEPD/SQNTLYFGAGTRLSVL | 89 |
| BV12-BJ1.2 | CASS/S/NSDYTFGSGTRLLVI | 90 |
| BV12-BJ1.3 | CASS/DRA/GNTLYFGEGSRLIVV | 91 |
| BV12-BJ1.5 | CAS S/WTG/NQAPLFGEGTRLSVL | 92 |
| BV12-BJ2.4 | CASS/LD/SQNTLYFGAGTRLSVL | 93 |
| BV12-BJ2.5 | CASS/LYE/DTQYFGPGTRLLVL | 94 |
| BV13-BJ1.1 | CAS S/PRDR/NTEVFFGKGTRLTVV | 95 |
| BV13-BJ1.4 | CASS/LQGD/NERLFFGHGTKLSVL | 96 |
| BV13-BJ2.5 | CASS/LWGD/QDTQYFGPGTRLLVL | 97 |
| BV14-BJ1.1 | CAWS/PPGT/NTEVFFGKGTRLTVV | 98 |
| BV14-BJ1.2 | CAWS/LPGQGD/SDYTFGSGTRLLVI | 99 |
| BV15-BJ2.1 | CGAR/DRQ/NYAEQFFGPGTRLTVL | 100 |
| BV15-BJ2.4 | CGAR/GR/QNTLYFGAGTRLSVL | 101 |
| BV16-BJ1.1 | CASS/QANK/EVFFGKGTRLTVV | 102 |
| BV18-BJ1.6 | CSS/NNRG/YNSPLYFAAGTRLTVT | 103 |
| BV18-BJ2.3 | CS/PRDWGA/SAETLYFGSGTRLTVL | 104 |
| BV20-BJ1.2 | CSSS/WDRA/SDYTFGSGTRLLVI | 105 |

TABLE 3
Amino acid sequences of beta CDR3s
over-represented in HY-incompatible skin
grafts at the time of rejection
by a single recipient (HY #1).

| BV-BJ Pair | BV/CDR3/BJ Sequence | SEQ ID NO: |
|---|---|---|
| BV1-BJ1.5 | CAS S/QEGGI/QAPLFGEGTRLSVL | 106 |
| BV1-BJ2.4 | CASS/QGGIN/QNTLYFGAGTRLSV | 107 |
| BV2-BJ1.3 | CSA/TEV/SGNTLYFGEGSRLIVV | 108 |
| BV2-BJ1.4 | CS/GNGQG/SNERLFFGHGTKLSVL | 109 |
| BV2-BJ1.5 | CSA/QG/NNQAPLFGEGTRLSVL | 110 |
| BV2-BJ2.4 | CRA/GRRG/SQNTLYFGAGTRLSVL | 111 |
| BV3-BJ1.1 | CASS/LSQ/NTEVFFGKGTRLTVV | 112 |
| BV3-BJ2.2 | CASS/RTD/TGQLYFGEGSKLTVL | 113 |
| BV3-BJ2.7 | CASS/LNRG/EQYFGPGTRLTVL | 114 |
| BV5.1-BJ2.1 | CASS/LNWGD/AEQFFGPGTRLTVL | 115 |
| BV5.1-BJ2.2 | CASS/LSGY/TGQLYFGEGSKLTVL | 116 |
| BV5.1-BJ2.5 | CASW/G/NQDTQYFGPGTRLLVL | 117 |
| BV5.2-BJ1.5 | CASS/PDS/NNQAPLFGEGTRLSVL | 118 |
| BV5.2-BJ2.3 | CASS/LGGA/SAETLYFGSGTRLTV | 119 |
| | L | |
| BV5.2-BJ2.5 | CASS/RTV/NQDTQYFGPGTRLLVL | 120 |
| BV6-BJ1.2 | CASS/MGQEA/SDYTFGSGTRLLVI | 121 |
| BV6-BJ2.1 | CASS/RGS/YAEQFFGPGTRLTVL | 122 |
| BV6-BJ2 .2 | CASS/LPGG/TGQLYFGEGSKLTVL | 123 |
| BV7-BJ1.1 | CASS/FSRS/NTEVFFGKGTRLTVV | 124 |
| BV7-BJ2.7 | CASS/WGWR/YEQYFGPGTRLTVL | 125 |
| BV8.1-BJ2.2 | CASS/DRSD/TGQLYFGEGSKLTVL | 126 |
| BV8.2-BJ1.4 | CASA/RDT/NERLFFGHGTKLSVL | 127 |
| BV8.2-BJ2.3 | CASG/GTT/SAETLYFGSGTRLTVL | 128 |
| BV8.3-BJ1.2 | CASS/DAH/SDYTFGSGTRLLVI | 129 |
| BV8.3-BJ1.5 | CASS/RES/NQAPLFGEGTRLSVL | 130 |
| BV8.3-BJ2.1 | CASS/DEDWA/YAEQFFGPGTRLTV | 131 |
| | L | |
| BV9-BJ1.4 | CASS/TGGAA/NERLFFGHGTKLSV | 132 |
| | L | |
| BV9-BJ2.3 | CASR/RRGR/AETLYFGSGTRLTVL | 133 |
| BV10-BJ1.5 | CASS/DRY/NNQAPLFGEGTRLSVL | 134 |
| BV10-BJ1.6 | CASR/RTF/SYNSPLYFAAGTRLTV | 135 |
| | T | |
| BV10-BJ2.7 | CASS/YP/YEQYFGPGTRLTVL | 136 |
| BV11-BJ1.3 | CASS/NRGL/GNTLYFGEGSRLIVV | 137 |
| BV11-BJ1.4 | CASS/LVERSK/ERLFFGHGTKLSV | 138 |
| | L | |
| BV11-BJ2.3 | CASR/AGGS/SAETLYFGSGTRLTV | 139 |
| | L | |
| BV12-BJ1.5 | CASS/LGR/NQAPLFGEGTRLSVL | 140 |
| BV12-BJ2.1 | CASS/LSGGD/AEQFFGPGTRLTVL | 141 |
| BV12-BJ2.4 | CASS/TQ/SQNTLYFGAGTRLSVL | 142 |
| BV13-BJ1.2 | CASS/LTG/NSDYTFGSGTRLLVI | 143 |
| BV13-BJ2.1 | CASS/FWGD/YAEQFFGPGTRLTVL | 144 |
| BV13-BJ2.5 | CASS/FTG/QDTQYFGPGTRLLVL | 145 |
| BV14-BJ1.5 | CAWR/QRV/NNQAPLFGEGTRLSVL | 146 |
| BV14-BJ1.6 | CAWS/RG/SYNSPLYFAAGTRLTVT | 147 |
| BV14-BJ2.1 | CAWS/RRV/NYAEQFFGPGTRLTVL | 148 |
| BV14-BJ2.5 | CAWS/LRLGA/QDTQYFGPGTRLLV | 149 |
| | L | |
| BV15-BJ1.3 | CGA/RDRVF/GNTLYFGEGSRLIVV | 150 |
| BV15-BJ1.4 | CGAG/QGT/NERLFFGHGTKLSVL | 151 |
| BV15-BJ1.6 | CGA/RDG/YNSPLYFAAGTRLTVT | 152 |
| BV18-BJ1.2 | CSSR/DSA/NSKYTFGSGTRLLVI | 153 |
| BV18-BJ1.4 | CSSR/GTGRG/ERLFFGHGTKLSVL | 154 |
| BV18-BJ2.1 | CSSR/ANS/YAEQFFGPGTRLTVL | 155 |
| BV18-BJ2.7 | CSSR/GGC/YEQYFGPGTRLTV | 156 |
| BV20-BJ1.1 | CSSS/LQG/TEVFFGKGTRLTVV | 157 |
| BV20-BJ1.6 | CSSS/QLAD/NSPLYFAAGTRLTVT | 158 |
| BV20-BJ2.5 | CSSS/QRTGGR/DTQYFGPGTRLLV | 159 |

### Example 2

### Analysis of Repertoires of T Cell Receptors in Human Cells

### Extraction of Total RNA

Total RNA was extracted from pelleted lymphocytes using an RNeasy Protect MiniKit (Qiagen) according to the manufacturer's instructions. Residual genomic DNA was removed from extracted RNA samples using an RNase-Free DNase Set (Qiagen).

### Primers

Primers were synthesized by the Invitrogen (Carlsbad, Calif.) SupplyCenter located at the Mayo Clinic Primer Core Facility (Rochester, Minn.). Sequences of 42 forward, outer primers were homologous to sequences within the CDR1 regions of BV genes (Table 4). These primers are divided into eight primer pools (designated in Table 4) for use in RT-PCRs with a biotinylated beta constant region primer. Forty-seven nested BV primers (Table 4) were based on sequences within the beta CDR2 regions, and each was paired with one of 13 BJ-specific primers (Table 5) to create a matrix of 611 fully-nested real time PCR reactions. Two RT-PCR and two nested PCR primers were designed for the purpose of normalizing the amounts of beta transcripts among samples and were based entirely on sequence within the beta constant region (Table 4).

TABLE 4
Human BV Primer sequences and Pool Distributions.

| | RT-PCR Primers | Pool | Seq | | Real Time PCR Primers | Seq |
|---|---|---|---|---|---|---|
| | (forward primer) | # | ID # | | (forward primers) | ID # |
| BV2 | GACAGGAAGTGATCTTGCGC | 3 | 161 | BV2 | AATCTTGGGGCAGAAAGTCG | 162 |
| BV3.1 | GATAATGTTTAGCTACAATAATAAGGAGC | 4 | 163 | BV3.1 | GATAATGTTTAGCTACAATAATAAGGAGC | 164 |
| BV4.1 | AGAAGTCTTTGAAATGTGAACAACATA | 4 | 165 | BV4.1 | TCATGTTTGTCTACAGCTATGAGAAA | 166 |
| BV4.2,3 | AACAACATCTGGGGCATAACG | 2 | 167 | BV4.2 | GAGCTCATGTTTGTCTACAACTTTAAA | 168 |
| BV5.1 | CCCTATCTCTGGGCATAGGAG | 1 | 169 | BV4.3 | AGCTCATGTTTGTCTACAGTCTTGAA | 170 |
| BV5.4 | CTTCTCAGTCTGGGCACAACAC | 2 | 171 | BV5.1 | TTCCTCTTTGAATACTTCAGTGAGAC | 172 |
| BV5.5 | TCTCCTATCTCTGGGCACAAGAG | 3 | 173 | BV5.4 | CAGTTTATCTTTCAGTATTATAGGGAGG | 174 |
| BV5.6 | TCTCCTAAGTCTGGGCATGACA | 1 | 175 | BV5.5 | CCCAGTTTATCTTTCAGTATTATGAGAA | 176 |
| BV5.8 | CCTATCTCTGGGCACACCAGT | 6 | 177 | BV5.6 | CCAGTTTATCTTTCAGTATTATGAGGAG | 178 |
| BV6.1 | TGCCCAGGATATGAACCATAACT | 2 | 179 | BV5.8 | CCTTTGGTATGACGAGGGTG | 180 |
| BV6.2, 3, 5 | GCCCAGGATATGAACCATGAA | 4 | 181 | BV6.1 | GATTTATTACTCAGCTTCTGAGGGT | 182 |
| BV6.4 | AGATGTACCCAGGATATGAGACATAAT | 1 | 183 | BV6.2, 3 | GCTGATTCATTACTCAGTTGGTGAG | 184 |
| BV6.6 | TGTACCCAGGATATGAACCATAACTA | 7 | 185 | BV6.4 | CTAAGGCTCATCCATTATTCAAATAC | 186 |
| BV6.8, 9 | CCCAGGATATGAACCATGGAT | 5 | 187 | BV6.5 | CTGATTCATTACTCAGTTGGTGCT | 188 |
| BV7.2, 3 | GGTGTGATCCAATTTCAGGTCATA | 6 | 189 | BV6.6 | GGGGCTGAAGCTGATTTATTAT | 190 |
| BV7.4 | TTCAATTTCGGGTCATGTAACC | 7 | 191 | BV6.8 | ACTACTCAGCTGCTGCTGGTACT | 192 |
| BV7.6, 8 | CCAATTTCGGGTCATGTATCC | 3 | 193 | BV6.9 | GCATGGGGCTGAGGCG | 194 |
| BV7.7 | GATCCAATTTCGAGTCATGCAA | 7 | 195 | BV7.2 | TTTTAATTTACTTCCAAGGCAACA | 196 |
| BV7.9 | ATCCAATTTCTGAACACAACCG | 8 | 197 | BV7.3 | TTCTAATTTACTTCCAAGGCACG | 198 |
| BV9 | TGCTCCCCTAGGTCTGGAGAC | 5 | 199 | BV7.4 | GGTTCTGACTTACTCCCAGAGTGA | 200 |
| BV10.1 | ACCAGACTTGGAACCACAACAAT | 2 | 201 | BV7.6 | TGACTTACTTCAATTATGAAGCCC | 202 |
| BV10.2 | CCAGACTTGGAGCCACAGCTAT | 1 | 203 | BV7.7 | CCCAGAGTTTCTGACTTACTTCAATTA | 204 |
| BV10.3 | ACCAGACTGAGAACCACCGC | 3 | 205 | BV7.8 | CTGACTTATTTCCAGAATGAAGCTC | 206 |
| BV11.1 | TGGCTTTTTGGTGTGATCCTAT | 5 | 207 | BV7.9 | GGGCCCAGAGTTTCTGACTTAC | 208 |
| BV11.2 | GGCTTTTTGGTGCAATCCTATA | 6 | 209 | BV9 | CAGTTCCTCATTCAGTATTATAATGGA | 210 |
| BV11.3 | GGCTTTTTGGTGCAATCCTATT | 8 | 211 | BV10.1 | TCCATTACTCATATGGTGTTCAAGA | 212 |
| BV12.3 | ACCAATTTCAGGCCACAACTC | 4 | 213 | BV10.2 | TCTATTACTCAGCAGCTGCTGATATT | 214 |
| BV12.4 | GTAAACCAATTTCAGGACACGACTA | 5 | 215 | BV10.3 | GATCCATTACTCATATGGTGTTAAAGA | 216 |
| BV12.5 | CAGCCAATTTTAGGCCACAATAC | 1 | 217 | BV11.1 | CCCGGAGCTTCTGGTTCAA | 218 |
| BV13 | CCACTCTGAAATGCTATCCTATCC | 3 | 219 | BV11.2 | CCAAAGCTTCTGATTCAGTTTCA | 220 |
| BV14 | GACCCAATTTCTGGACATGATAAT | 7 | 221 | BV11.3 | GATTCGATATGAGAATGAGGAAGC | 222 |
| BV15 | GTTCTCAGACTTTGAACCATAACGT | 7 | 223 | BV12.3 | ATTTACTTTAACAACAACGTTCCG | 224 |
| BV16 | CAAAATTATATTGTGCCCCAATAA | 6 | 225 | BV12.4 | ATTTACTTTAACAACAACGTTCCG | 226 |
| BV18 | CCCAATGAAAGGACACAGTCAT | 2 | 227 | BV12.5 | ACTTCCGCAACCGGGCT | 228 |
| BV19 | TGAACAGAATTTGAACCACGATG | 1 | 229 | BV13 | ATTTCGTTTTATGAAAAGATGCAG | 230 |
| BV20 | CTGTGAAGATCGAGTGCCGTT | 6 | 231 | BV14 | TCTGTTACATTTTGTGAAAGAGTCTAAA | 232 |
| BV24 | TGTTCTCAGACTAAGGGTCATGATAG | 4 | 233 | BV15 | AAAGCTGCTGTTCCACTACTATGA | 234 |
| BV25 | CACTCTGGAATGTTCTCAAACCA | 8 | 235 | BV16 | ATTTCCTTCCAGAATGAAAATGTC | 236 |
| BV27 | TTGTTCTCAGAATATGAACCATGAGTAT | 5 | 237 | BV18 | ATGGTTTATCTCCAGAAAGAAAATATC | 238 |
| BV28 | GGAATGTGTCCAGGATATGGAC | 8 | 239 | BV19 | ATTGATCTACTACTCACAGATAGTAAATGAC | 240 |
| BV29 | AAGTCGATAGCCAAGTCACCAT | 7 | 241 | BV20 | CAACTTCCAATGAGGGCTCC | 242 |
| BV30 | GTGGAGGGAACATCAAACCC | 8 | 243 | BV24 | GCCTACGGTTGATCTATTACTCCT | 244 |
| BV25 | | | | | CTCATCCACTATTCCTATGGAGTTAA | 245 |
| BV27 | | | | | AGATCTACTATTCAATGAATGTTGAGG | 246 |
| BV28 | | | | | GCTGATCTATTTCTCATATGATGTTAAAAT | 247 |
| BV29 | | | | | TGACACTGATCGCAACTGCAA | 248 |
| BV30 | | | | | CTTCTACTCCGTTGGTATTGGC | 249 |

TABLE 5
Human BJ and TCRBC control primers.

| BJ primers | | Seq ID |
|---|---|---|
| (reverse primers) | | #: |
| BJ1.1 | TGCCTTGTCCAAAGAAAGCT | 250 |
| BJ1.2 | CGAACCGAAGGTGTAGCCA | 251 |
| BJ1.3 | AACTTCCCTCTCCAAAATATATGGT | 252 |
| BJ1.4 | TTCCACTGCCAAAAAACAGTTT | 253 |
| BJ1.5 | ACCAAAATGCTGGGGCTG | 254 |
| BJ1.6 | CCCAAAGTGGAGGGGTGAA | 255 |
| BJ2.1 | CCCGAAGAACTGCTCATTGT | 256 |
| BJ2.2 | CCTTCTCCAAAAAACAGCTCC | 257 |
| BJ2.3 | CTGGGCCAAAATACTGCGTA | 258 |
| BJ2.4 | CGGCGCCGAAGTACTGAAT | 259 |
| BJ2.5 | TGGCCCGAAGTACTGGGTC | 260 |
| BJ2.6 | GCCCCGAAAGTCAGGACG | 261 |
| BJ2.7 | GGCCCGAAGTACTGCTCGTA | 262 |

### Constant Region Control Primers

TCRBC RT-PCR primers:

| | |
|---|---|
| CCGAGGTCGCTGTGTTTGAG (forward) | (SEQ ID NO: 263) |
| Bio-GGACTTGACAGCCGAAGTGG (reverse) | (SEQ ID NO: 264) |
| TCRBC Real Time PCR primers: | |
| CAAAAGGCCACACTGGTGTG (forward) | (SEQ ID NO: 265) |
| CTGCTCAGGCAGTATCTGGAG (reverse) | (SEQ ID NO: 266) |

### RT-PCRs

Eight pooled RT-PCRs were performed in 50 μL volumes using a One-Step RT-PCR Kit (Qiagen), 21 ng of total RNA, 20 pmol of a 5′-biotinylated BC primer, and pools of BV primers (six pools of five primers and two pools of six primers) that provided 6.6 pmol of each BV primer. One additional RT-PCR reaction was performed using 20 pmol of each of the RT-PCR primers that were based on sequence in the constant region. RNA templates were denatured at 75° C. for 4 minutes and placed on ice prior to addition to RT-PCR reactions. Cycling was performed on a PTC-225 Peltier Thermal Cycler (MJ Research, Waltham, Mass.) as follows: cDNA synthesis was performed at 50° C. for 32 minutes followed by incubation at 95° C. for 15 minutes to inactivate the reverse transcriptase. Subsequent PCR parameters were 1 minute at 94° C., 30 seconds at 60° C., and 1 minute at 72° C. for 25 cycles. A final extension cycle was performed for 6 minutes at 72° C. RT-PCR products were separated from residual primers and amplification reagents using a QIAquick PCR Purification Kit (Qiagen) and eluted with 50 μL of elution buffer.

### Enrichment of Biotinylated PCR Products

Biotinylated RT-PCR products were purified with My One™ Streptavidin C1 Dynabeads (Dynal Biotech ASA, Oslo, Norway) following the manufacturer's protocol. Briefly, 50 μL of Dynabeads were washed two times in 50 μL of 2× washing and binding buffer (10 mM Tris-HCl (pH 7.5), 1 mM EDTA, and 2 M NaCl). Following the second wash, the beads were resuspended in 100 μL of 2× washing and binding buffer, 50 μL of PCR product, and 50 μL of sterile water. The suspensions were incubated for 15 minutes at room temperature with gentle shaking. The amplicon-bound beads were washed twice with 100 μL of 1× washing and binding buffer and then resuspended in 100 μL of 10 mM Tris-HCl, pH 8.5. Suspensions of amplicon-bound beads were diluted 1:10 for direct use as templates in real time PCR reactions.

### Real Time PCR

A total of 611 individual real-time PCRs (47 BV and 13 BJ primers) were performed in 10 μL volumes in 384-well Clear Optical Reaction Plates with Optical Adhesive Covers (Applied Biosystems, Foster City, Calif.). The components of reactions were 10 pmol of a nested BV primer (Table 4), 10 pmol of a BJ-specific primer (Table 5), 1 μL of the respective amplicon-bound bead suspension, and 5 μL Power SYBR Green PCR Master Mix (2×) (Applied Biosystems). One additional reaction was performed using 10 pmol of each the nested constant region primers and 1 μL of the respective amplicon-bound bead suspension. Cycling was performed on an ABI Prism 7900HT Sequence Detection System at the AGTC Microarray Shared Resource Core Facility (Mayo Clinic) using SYBR Green detection. Cycling parameters were as follows: (1) an initial incubation at 50° C. for 2 minutes, (2) a 10 minute incubation at 95° C. to activate the DNA polymerase, and (3) 40 cycles of 15 seconds at 95° C. followed by 1 minute at 60° C. Dissociation curves were generated by (1) incubating the amplicons at 95° C. for 15 seconds, (2) reducing the temperature to 60° for 15 seconds, and (3) increasing the temperature to 95° C. over a dissociation time of 20 minutes. Data were analyzed with the 7900HT SequenceDetectionSystem (SDS) Version 2.3 software (Applied Biosystems) to estimate cycle threshold (Ct) values and dissociation curves to estimate the optimal melting temperatures for all reactions. Ct values were fractional cycle numbers at which fluorescence passes the threshold level (designated by a horizontal line in Ct plots), that is automatically set to be within the exponential region of the amplification curve where there is a linear relationship between the log of change in fluorescence and cycle number. Dissociation curves were formed by plotting rising temperatures versus the change in fluorescence/change in temperature.

### Statistical Analysis

Wilcoxon matched pairs and Kruskal-Wallis tests were used to estimate the statistical significance of differences in representation of BV-BJ combinations. The relative abundance of BV-BJ combinations was defined by the observed Ct values and dissociation curves. Dissociation curves were used to confirm the presence of amplicons from beta transcripts by excluding (1) primer-dimers that have relatively low melting temperatures and (2) amplicons with peak heights that do not exceed a threshold of 0.07 (change in fluorescence/change in temperature). This threshold was selected due to the inability to sequence amplicons that are below this value. Amplicons with either or both of these characteristics were assigned Ct values of >40 cycles. Arrays of Ct values were paired according to BV-BJ combinations and Wilcoxon matched pairs, and Kruskal-Wallis tests were performed with Prism software (GraphPad Software, San Diego, Calif.).

### Results

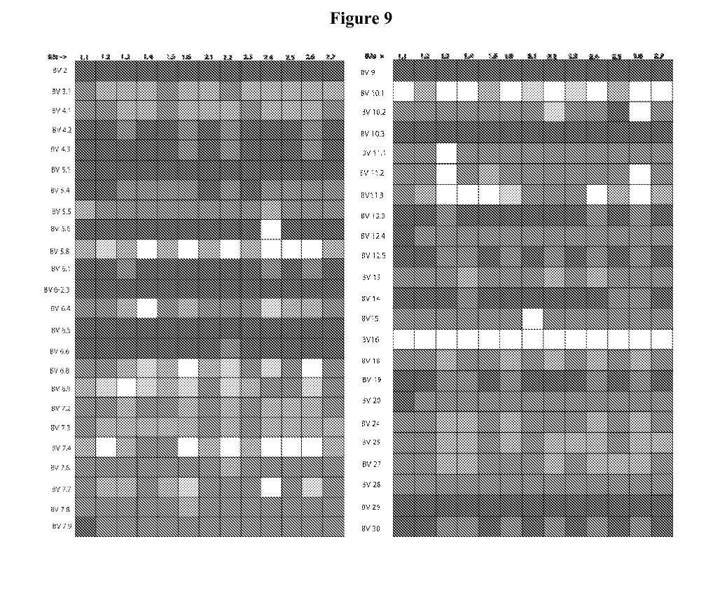

A test of the human BV-BJ matrix method was performed with RNA extracted from a cord blood sample. Pooled RT-PCRs and real-time PCR were performed, and the results are summarized in FIG. 9. Products were generated for all tested BV genes with the exception of BV16. An analysis of a second cord blood sample resulted in two strong BV16-BJ products which demonstrated that the BV 16 primer pair was functional and that the first sample simply lacked BV16 transcripts. Taking the Ct values and confirmation of specific amplification with dissociation curves into consideration, about 95% of the BV-BJ primer combinations yielded specific products. The Ct values estimated with two human T cell repertoires were increased by about one to two cycles when compared to values obtained with murine samples with equivalent amounts of RNA. This is consistent with the 2.5-fold greater diversity of BV-BJ transcripts expressed in human lymphocyte populations.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing T cell receptor diversity in a mammal, wherein said method comprises performing a real-time amplification reaction using a BV-specific primer, a BJ-specific primer, and sample of nucleic acid containing template, wherein said sample is enriched to contain BV-BC nucleic acid sequences.

2. The method of claim 1, wherein said mammal is a human.

3. The method of claim 1, wherein said sample was enriched using an amplification reaction that amplifies BV-BC nucleic acid sequences.

4. The method of claim 3, wherein said amplification reaction that amplifies BV-BC nucleic acid sequences comprises using an outer BV-specific primer and a BC-specific primer, wherein one of said outer BV-specific primer and said BC-specific primer comprises a label.

5. The method of claim 4, wherein said label comprises biotin.

6. The method of claim 5, wherein streptavidin-containing magnetic particles are used to enrich said sample.

7. The method of claim 1, wherein said method comprises performing said real-time amplification reaction using a collection of different BV-specific primers, a collection of different BJ-specific primers, and said sample.

8. The method of claim 1, wherein said collection of different BV-specific primers comprises a primer specific for each BV nucleic acid present is said mammal.

9. The method of claim 1, wherein said collection of different BJ-specific primers comprises a primer specific for each BJ nucleic acid present is said mammal.

10. The method of claim 1, wherein said sample was enriched using pools of amplification reactions that amplify BV-BC nucleic acid sequences.
